(19) United States
(12) Patent Application Publication
(10) Pub. No.: US 2011/0171200 A1
(43) Pub. Date: Jul. 14, 2011

(54) PROTEIN C RS2069915 AS A RESPONSE PREDICTOR TO SURVIVAL AND ADMINISTRATION OF ACTIVATED PROTEIN C OR PROTEIN C-LIKE COMPOUND

(76) Inventors: Keith R. Walley, North Vancouver (CA); James A. Russell, Vancouver (CA); Asim Sarosh Siddiqui, San Francisco, CA (US); Anthony Gordon, London (GB); Mark D. Williams, Indianapolis, IN (US); William L. Macias, Indianapolis, IN (US); Sandra Close Kirkwood, Indianapolis, IN (US)

(21) Appl. No.: 12/812,934

(22) PCT Filed: Jan. 15, 2009
(86) PCT No.: PCT/CA09/00037

§ 371 Date: Mar. 11, 2011

Related U.S. Application Data
(60) Provisional application No. 61/006,456, filed on Jan. 15, 2008.

(51) Int. Cl. C12Q 1/68; A61K 38/48; A61K 38/02; C40B 30/04; C40B 40/06; C40B 40/10; A61P 5/00; A61P 29/00; H01J 49/40

(52) U.S. Cl. 424/94.64; 435/6.11; 514/1.4; 506/9; 506/16; 506/18; 250/287

(57) ABSTRACT

Provided herein are methods, oligonucleotides and peptide nucleic acids, compositions and kits for predicting a subject's response to treatment with activated protein C or protein C-like compound or susceptibility to major organ dysfunction or susceptibility to an inflammatory condition. The method generally comprises determining a genotype of said subject at one or more of polymorphic sites in the subject's protein C gene selected from one or more of the following: rs20069915 and one or more polymorphism sites in linkage disequilibrium thereto, selected from one or more of the following: rs2069910; rs2069916; rs2069924; rs2069931; rs1799808; rs2069920; and rs6714364 and may further involve comparing the determined genotype with known genotypes for the polymorphism that correspond with an improved response to treatment with activated protein C or protein C-like compound or correspond to susceptibility to major organ dysfunction or susceptibility to an inflammatory condition. Also provided are methods of treating subjects with an anti-inflammatory agent or anti-coagulant agent based on the subject's genotype.

### FIELD OF INVENTION

The field of invention relates to the assessment and/or treatment of subjects with an inflammatory condition.

### BACKGROUND OF THE INVENTION

Recent studies have demonstrated a relationship between genotype and response to pharmacological therapeutics (ie. pharmacogenomics). Genentech's HERCEPTIN® was not effective in its overall Phase III trial but was shown to be of therapeutic benefit in a genetic subset of patients with human epidermal growth factor receptor 2 (HER2)-positive metastatic breast cancer. Similarly, Novartis' GLEEVEC® is only indicated for the subset of chronic myeloid leukemia patients who carry a reciprocal translocation between chromosomes 9 and 22 (i.e. the Philadelphia chromosome).

The septic inflammatory response involves complex cross-talk within and between the inflammation, coagulation and apoptosis pathways. Homeostatic imbalance of these and other counter-regulatory pathways can lead to altered clinical outcome in subjects with inflammatory conditions such as severe sepsis. Naturally-occurring genetic variation in human populations is one mechanism that can induce such a response. Furthermore, the genotype of an individual has been demonstrated to predict clinical outcome with respect to various inflammatory and infectious phenotypes (ARCAROLI J et al. Shock (2005) 24(4):300-12; SUTHERLAND A M et al. Crit. Care Med (2005) 33(3):638-44; WATANABE E et al. J Trauma (2005) 59(5):1181-9; GORDON A C et al. Shock (2006) 25(1):88-93).

The human Protein C gene (PROC) maps to chromosome 2q13-q14. A representative Homo sapiens PROC gene sequence is listed in GenBank under accession number NM 000312 (GI:109389366). PROC encodes a precursor protein consisting of 461 amino acids. Protein C is synthesized primarily in the liver and secreted into the plasma where it exists in its inactive form until it is cleaved by the thrombin:thrombomodulin complex. Activated Protein C (APC) modulates the coagulation cascade by inactivating coagulation factor Va (WALKER F J. et al. Biochim Biophys Acta (1979) 571(2):333-42) and coagulation factor VIIIa (FULCHER C A. et al. Blood (1984) 63(2):486-9). APC also attenuates the synthesis of plasminogen activator inhibitor type 1 (SERPINE1) (VAN HINSBERGH V W. et al. Blood (1985) 65(2):444-51).

APC demonstrates anti-inflammatory activity through binding to the Protein C Receptor (PROCR) to activate the Factor 2 Receptor (F2R or PAR1; RIEWALD M. et al. Science (2002) 296(5574):1880-2). F2R is a G protein-coupled receptor whose activation decreases downstream NFκB signaling and subsequent TNFα, IL1β, and IL6 expression (GREY S T. et al. Journal of Immunology (1994) 153(8):3664-72; HANCOCK W W. et al. Transplantation (1995) 60(12):1525-32; and MURAKAMI K. et al. American Journal of Physiology (1997) 272(2 Pt 1):L197-202). APC also decreases neutrophil adhesion to endothelial cells, decreases neutrophil chemotaxis and decreases apoptosis of endothelial cells and neurons (GRINNELL B W. et al. Glycobiology (1994) 4(2):221-5; JOYCE D E. et al. J Biol Chem (2001) 276(14):11199-203; STURN D H. et al. Blood (2003) 102(4):1499-505; and LIU D. et al. Nat Med (2004) 10(12):1379-83). Accordingly, APC has been implicated as having a central role in the pathophysiology of the systemic inflammatory response syndrome and the inflammatory sequelae arising from sepsis.

Decreased plasma levels of protein C are observed in association with the inflammatory response arising from sepsis, major surgery, or shock (GRIFFIN J H. et al. Blood (1982) 60(1):261-4; BLAMEY S L. et al. Thromb Haemost (1985) 54(3):622-5; TAYLOR F B. et al. Journal of Clinical Investigation (1987) 79(3):918-25; HESSELVIK J F. et al. Thromb Haemost (1991) 65(2):126-9; FIJNVANDRAAT K. et al. Thromb Haemost (1995) 73(1):15-20; and FAUST S N. et al. N Engl J Med (2001) 345(6):408-16) and is related to poor outcome (LORENTE J A. et al. Chest (1993) 103(5):1536-42; VERVLOET M G. et al. Semin Thromb Hemost (1998) 24(1):33-44; FISHER C J. Jr. and YAN S B. Crit. Care Med (2000) 28(9 Suppl):S49-56; YAN S B. and DHAINAUT J F. Crit. Care Med (2001) 29(7 Suppl):S69-74; and LAY A J. et al. Blood (2006; Epub ahead of print). The expression of endothelial cell proteins such as thrombomodulin and protein C receptor (PROCR) is also impaired by pro-inflammatory cytokines and thus may also serve as a mechanism by which Protein C function is abrogated (STEARNS-KUROSAWA D J. et al. Proceedings of the National Academy of Sciences of the United States of America (1996) 93(19):10212-6).

Therapeutic agents for severe sepsis often target one or more of the pathways intrinsic to inflammation and infection. In particular, XIGRIS™ (drotrecogin alpha activated, activated protein C, APC) having anti-inflammatory, anti-coagulant, pro-fibrinolytic and anti-apoptotic activity, has been observed to decrease 28-day mortality in both experimental sepsis models (LAY A J et al. Blood (2006; Epub ahead of print) and in the Phase III PROWESS severe sepsis trial (BERNARD G R. et al. New England Journal of Medicine (2001) 344(10):699-709; MACIAS W L et al. Crit. Care (2005) 9(Suppl4):S38-45).

### SUMMARY OF THE INVENTION

In accordance with one aspect of the invention, methods are provided for identifying a subject predisposed or susceptible to major organ dysfunction, the method comprising determining a genotype of said subject at one or more of polymorphic sites in the subject's protein C gene selected from: rs2069915 and one or more polymorphic sites in linkage disequilibrium thereto.

In accordance with another aspect of the invention, methods are provided for identifying a polymorphism in a protein C gene sequence that correlates with an improved response to activated protein C or protein C like compound administration, the method including:
a) obtaining protein C gene sequence information from a group of subjects having an inflammatory condition;
b) identifying at least one polymorphic nucleotide position in the protein C gene sequence in the subjects selected from the following: rs2069915; rs2069910; rs2069916; rs2069924; rs2069931; rs1799808; rs2069920; and rs6714364;
c) determining a genotype at the polymorphic site for individual subjects in the group;
d) determining response to activated protein C or protein C like compound administration; and
e) correlating the genotypes determined in step (c) with the response to activated protein C or protein C like compound administration in step (d) thereby identifying said protein C gene sequence polymorphisms that correlate with response to activated protein C or protein C like compound administration.

In accordance with another aspect of the invention, kits and commercial packages are provided for determining a genotype at a defined nucleotide position within a polymorphic site in a protein C gene sequence in a subject to predict a subject's response to activated protein C or protein C like compound administration, the kit or commercial package may include: a restriction enzyme capable of distinguishing alternate nucleotides at the polymorphic site selected from the following: rs2069915; rs2069910; rs2069916; rs2069924; rs2069931; rs1799808; rs2069920; and rs6714364; or a labeled oligonucleotide having sufficient complementary to the polymorphic site selected from the following: rs2069915; rs2069910; rs2069916; rs2069924; rs2069931; rs1799808; rs2069920; and rs6714364, so as to be capable of hybridizing distinctively to said alternate. The kit or commercial package may further include an oligonucleotide or a set of oligonucleotides operable to amplify a region including the polymorphic site. The kit or commercial package may further include a polymerization agent. The kit or commercial package may further include instructions for using the kit or commercial package to determine genotype.

In accordance with another aspect of the invention, methods are provided for selecting a group of subjects for determining the efficacy of a candidate drug known or suspected of being useful for the treatment of an inflammatory condition, the method may include determining a genotype at one or more of the following polymorphic sites: rs2069915; rs2069910; rs2069916; rs2069924; rs2069931; rs1799808; rs2069920; and rs6714364, for each subject, wherein the genotype is indicative of the subject's response to the candidate drug and sorting subjects based on their genotype. The method may further include, administering the candidate drug to the subjects or a subset of subjects and determining each subject's ability to recover from the inflammatory condition. The method may further include comparing subject response to the candidate drug based on genotype of the subject.

In accordance with another aspect of the invention, methods are provided for treating an inflammatory condition in a subject in need thereof, the method may include administering to the subject activated protein C or protein C like compound, wherein said subject has an improved response polymorphism at one or more of the following sites: rs2069915; rs2069910; rs2069916; rs2069924; rs2069931; rs1799808; rs2069920; and rs6714364.

In accordance with another aspect of the invention, methods are provided for treating an inflammatory condition in a subject in need thereof, the method may include: selecting a subject having an improved response polymorphism at one or more of the following sites: rs2069915; rs2069910; rs2069916; rs2069924; rs2069931; rs1799808; rs2069920; and rs6714364; and administering to said subject activated protein C or protein C like compound.

In accordance with another aspect of the invention, methods are provided for treating a subject with an inflammatory condition by administering activated protein C, the method may include administering the activated protein C or protein C like compound to subjects that have an improved response polymorphism at one or more of the following sites: rs2069915; rs2069910; rs2069916; rs2069924; rs2069931; rs1799808; rs2069920; and rs6714364, wherein the improved response polymorphism is predictive of increased responsiveness to the treatment of the inflammatory condition with activated protein C or protein C like compound.

In accordance with another aspect of the invention, methods are provided for selecting a subject for the treatment of an inflammatory condition with an activated protein C or protein C like compound, including the step of identifying a subject having an improved response polymorphism at one or more of the following sites: rs2069915; rs2069910; rs2069916; rs2069924; rs2069931; rs1799808; rs2069920; and rs6714364, wherein the identification of a subject with the improved response polymorphism is predictive of increased responsiveness to the treatment of the inflammatory condition with the activated protein C or protein C like compound.

In accordance with another aspect of the invention, methods are provided for treating an inflammatory condition in a subject, the method may include administering an activated protein C or protein C like compound to the subject, wherein said subject has an improved response polymorphism at one or more of the following sites: rs2069915; rs2069910; rs2069916; rs2069924; rs2069931; rs1799808; rs2069920; and rs6714364.

In accordance with another aspect of the invention, methods are provided for treating an inflammatory condition in a subject, the method may include: identifying a subject having an improved response polymorphism at one or more of the following sites: rs2069915; rs2069910; rs2069916; rs2069924; rs2069931; rs1799808; rs2069920; and rs6714364; and administering activated protein C or protein C like compound to the subject.

In accordance with another aspect of the invention, uses are provided for the manufacture of a medicament with activated protein C or protein C like compound for the treatment of an inflammatory condition, wherein the subjects treated have an improved response at one or more of the following sites: rs2069915; rs2069910; rs2069916; rs2069924; rs2069931; rs1799808; rs2069920; and rs6714364.

In accordance with another aspect of the invention, uses are provided for the manufacture of a medicament with activated protein C or protein C like compound for the treatment of an inflammatory condition in a subset of subjects, wherein the subset of subjects have an improved response polymorphism at one or more of the following sites: rs2069915; rs2069910; rs2069916; rs2069924; rs2069931; rs1799808; rs2069920; and rs6714364.

The method or use may further include determining the subject's APACHE II score as an assessment of subject risk. The method or use may further include determining the number of organ system failures for the subject as an assessment of subject risk. A subject's APACHE II score may be indicative of an increased risk when ≧25. Two or more organ system failures may be indicative of increased subject risk.

In accordance with another aspect of the invention, methods are provided for identifying a subject predisposed or susceptible to an inflammatory condition, the method may include determining a genotype of the subject at one or more polymorphic sites in the subject's protein C gene selected from: rs2069915 and one or more polymorphic sites in linkage disequilibrium thereto.

In accordance with another aspect of the invention, two or more oligonucleotides or peptide nucleic acids of about 10 to about 400 nucleotides are provided that hybridize specifically to a sequence contained in a human target sequence consisting of a subject's protein C gene sequence, a complementary sequence of the target sequence or RNA equivalent of the target sequence and wherein the oligonucleotides or peptide nucleic acids are operable in determining the presence or absence of two or more polymorphisms in their protein C gene sequence selected from of the following polymorphic sites: rs2069915; rs2069910; rs2069916; rs2069924; rs2069931; rs1799808; rs2069920; and rs6714364. The polymorphisms may be improved response polymorphisms.

In accordance with another aspect of the invention, two or more oligonucleotides or peptide nucleic acids are provided which may be selected from the group consisting of:

(a) an oligonucleotide or peptide nucleic acid that hybridizes under high stringency conditions to a nucleic acid molecule comprising SEQ ID NO:1 having a G at position 301 but not to a nucleic acid molecule comprising SEQ ID NO:1 having a A at position 301;
(b) an oligonucleotide or peptide nucleic acid that hybridizes under high stringency conditions to a nucleic acid molecule comprising SEQ ID NO:1 having an A at position 301 but not to a nucleic acid molecule comprising SEQ ID NO:1 having a G at position 301;
(c) an oligonucleotide or peptide nucleic acid that hybridizes under high stringency conditions to a nucleic acid molecule comprising SEQ ID NO:2 having a C at position 301 but not to a nucleic acid molecule comprising SEQ ID NO:2 having a T at position 301;
(d) an oligonucleotide or peptide nucleic acid that hybridizes under high stringency conditions to a nucleic acid molecule comprising SEQ ID NO:2 having a T at position 301 but not to a nucleic acid molecule comprising SEQ ID NO:2 having a C at position 301;
(e) an oligonucleotide or peptide nucleic acid that hybridizes under high stringency conditions to a nucleic acid molecule comprising SEQ ID NO:3 having a C at position 301 but not to a nucleic acid molecule comprising SEQ ID NO:3 having a T at position 301;
(f) an oligonucleotide or peptide nucleic acid that hybridizes under high stringency conditions to a nucleic acid molecule comprising SEQ ID NO:3 having a T at position 301 but not to a nucleic acid molecule comprising SEQ ID NO:3 having a C at position 301;
(g) an oligonucleotide or peptide nucleic acid that hybridizes under high stringency conditions to a nucleic acid molecule comprising SEQ ID NO:4 having a C at position 301 but not to a nucleic acid molecule comprising SEQ ID NO:4 having a T at position 301;
(h) an oligonucleotide or peptide nucleic acid that hybridizes under high stringency conditions to a nucleic acid molecule comprising SEQ ID NO:4 having a T at position 301 but not to a nucleic acid molecule comprising SEQ ID NO:4 having a C at position 301;
(i) an oligonucleotide or peptide nucleic acid that hybridizes under high stringency conditions to a nucleic acid molecule comprising SEQ ID NO:5 having a C at position 301 but not to a nucleic acid molecule comprising SEQ ID NO:5 having a T at position 301;
(j) an oligonucleotide or peptide nucleic acid that hybridizes under high stringency conditions to a nucleic acid molecule comprising SEQ ID NO:5 having a T at position 301 but not to a nucleic acid molecule comprising SEQ ID NO:5 having a C at position 301;
(k) an oligonucleotide or peptide nucleic acid that hybridizes under high stringency conditions to a nucleic acid molecule comprising SEQ ID NO:6 having a C at position 433 but not to a nucleic acid molecule comprising SEQ ID NO:6 having a T at position 433;
(l) an oligonucleotide or peptide nucleic acid that hybridizes under high stringency conditions to a nucleic acid molecule comprising SEQ ID NO:6 having a T at position 433 but not to a nucleic acid molecule comprising SEQ ID NO:6 having a C at position 433;
(m) an oligonucleotide or peptide nucleic acid that hybridizes under high stringency conditions to a nucleic acid molecule comprising SEQ ID NO:7 having a C at position 201 but not to a nucleic acid molecule comprising SEQ ID NO:7 having a T at position 201;
(n) an oligonucleotide or peptide nucleic acid that hybridizes under high stringency conditions to a nucleic acid molecule comprising SEQ ID NO:7 having a T at position 201 but not to a nucleic acid molecule comprising SEQ ID NO:7 having a C at position 201;
(o) an oligonucleotide or peptide nucleic acid that hybridizes under high stringency conditions to a nucleic acid molecule comprising SEQ ID NO:8 having a T at position 326 but not to a nucleic acid molecule comprising SEQ ID NO:8 having an A at position 326; and
(p) an oligonucleotide or peptide nucleic acid that hybridizes under high stringency conditions to a nucleic acid molecule comprising SEQ ID NO:8 having an A at position 326 but not to a nucleic acid molecule comprising SEQ ID NO:8 having a T at position 326.

In accordance with another aspect of the invention, an array of oligonucleotides or peptide nucleic acids attached to a solid support are provided, the array may include two or more of the oligonucleotides or peptide nucleic acids set out herein. The oligonucleotides or peptide nucleic acids may be attached to the solid support through a linker molecule.

In accordance with another aspect of the invention, a composition including an addressable collection of two or more oligonucleotides or peptide nucleic acids are provided, the two or more oligonucleotides or peptide nucleic acids selected from the oligonucleotides or peptide nucleic acids set out herein.

In accordance with another aspect of the invention, a composition including an addressable collection of two or more oligonucleotides or peptide nucleic acids are provided, wherein the two or more oligonucleotides or peptide nucleic acids may include two or more nucleic acid molecules set out in SEQ ID NO:1-8 or compliments, fragments, variants, or analogs thereof.

In accordance with another aspect of the invention, a composition including an addressable collection of two or more oligonucleotides or peptide nucleic acids is provided, the two or more oligonucleotides or peptide nucleic acids may be selected from two or more nucleic acid molecules set out in TABLES 1C and 1D or compliments, fragments, variants, or analogs thereof.

The oligonucleotides or peptide nucleic acids may further include one or more of the following: a detectable label; a quencher; a mobility modifier; a contiguous non-target sequence situated 5′ or 3′ to the target sequence or 5′ and 3′ to the target sequence.

In accordance with another aspect of the invention, a computer readable medium including a plurality of digitally encoded genotype correlations selected from the protein C gene SNP correlations in TABLE 1E, wherein each correlation of the plurality has a value representing an indication of responsiveness to treatment with activated protein C.

The polymorphic site in linkage disequilibrium thereto may be selected from one or more of the following: rs2069910; rs2069916; rs2069924; rs2069931; rs1799808; rs2069920; and rs6714364. The subject may be human. The subject may be critically ill with an inflammatory condition.

The inflammatory condition may be selected from the group consisting of: sepsis, septicemia, pneumonia, septic shock, systemic inflammatory response syndrome (SIRS), Acute Respiratory Distress Syndrome (ARDS), acute lung injury, aspiration pneumanitis, infection, pancreatitis, bacteremia, peritonitis, abdominal abscess, inflammation due to trauma, inflammation due to surgery, chronic inflammatory disease, ischemia, ischemia-reperfusion injury of an organ or tissue, tissue damage due to disease, tissue damage due to chemotherapy or radiotherapy, and reactions to ingested, inhaled, infused, injected, or delivered substances, glomerulonephritis, bowel infection, opportunistic infections, and for subjects undergoing major surgery or dialysis, subjects who are immunocompromised, subjects on immunosuppressive agents, subjects with HIV/AIDS, subjects with suspected endocarditis, subjects with fever, subjects with fever of unknown origin, subjects with cystic fibrosis, subjects with diabetes mellitus, subjects with chronic renal failure, subjects with acute renal failure, oliguria, subjects with acute renal dysfunction, glomerulo-nephritis, interstitial-nephritis, acute tubular necrosis (ATN), subjects, subjects with bronchiectasis, subjects with chronic obstructive lung disease, chronic bronchitis, emphysema, or asthma, subjects with febrile neutropenia, subjects with meningitis, subjects with septic arthritis, subjects with urinary tract infection, subjects with necrotizing fasciitis, subjects with other suspected Group A streptococcus infection, subjects who have had a splenectomy, subjects with recurrent or suspected enterococcus infection, other medical and surgical conditions associated with increased risk of infection, Gram positive sepsis, Gram negative sepsis, culture negative sepsis, fungal sepsis, meningococcemia, post-pump syndrome, cardiac stun syndrome, myocardial infarction, stroke, congestive heart failure, hepatitis, epiglotittis, E. coli 0157:H7, malaria, gas gangrene, toxic shock syndrome, pre-eclampsia, eclampsia, HELP syndrome, mycobacterial tuberculosis, Pneumocystic carinii, pneumonia, Leishmaniasis, hemolytic uremic syndrome/thrombotic thrombocytopenic purpura, Dengue hemorrhagic fever, pelvic inflammatory disease, Legionella, Lyme disease, Influenza A, Epstein-Barr virus, encephalitis, inflammatory diseases and autoimmunity including Rheumatoid arthritis, osteoarthritis, progressive systemic sclerosis, systemic lupus erythematosus, inflammatory bowel disease, idiopathic pulmonary fibrosis, sarcoidosis, hypersensitivity pneumonitis, systemic vasculitis, Wegener's granulomatosis, transplants including heart, liver, lung kidney bone marrow, graft-versus-host disease, transplant rejection, sickle cell anemia, nephrotic syndrome, toxicity of agents such as OKT3, cytokine therapy, cirrhosis, disseminated intravascular coagulation (DIC), cardiogenic shock, and acute kidney injury. The inflammatory condition may be SIRS. The inflammatory condition may be sepsis. The inflammatory condition may be septic shock.

The method may further include obtaining protein C gene sequence information for the subject. The genotype may be determined using a nucleic acid sample from the subject. The method may further include obtaining the nucleic acid sample from the subject. The genotype may be determined using one or more of the following techniques: restriction fragment length analysis; sequencing; micro-sequencing assay; hybridization; invader assay; gene chip hybridization assays; oligonucleotide ligation assay; ligation rolling circle amplification; 5′ nuclease assay; polymerase proofreading methods; allele specific PCR; matrix assisted laser desorption ionization time of flight (MALDI-TOF) mass spectroscopy; ligase chain reaction assay; enzyme-amplified electronic transduction; single base pair extension assay; and reading sequence data.

The method may include administration of an activated protein C or protein C like compound according to genotype.

The genotype may be rs2069915 AA or rs2069915 GG or one or more polymorphic sites in linkage disequilibrium thereto. The genotype of one or more polymorphic sites in linkage disequilibrium thereto may be selected from the following: rs2069910 CC or rs2069910 TT; rs2069916 CC or rs2069916 TT; rs2069924 CC or rs2069924 TT; rs2069931 CC or rs2069931 TT; rs1799808 CC or rs1799808 TT; rs2069920 CC or rs2069920 TT; and rs671464 AA or rs671464 TT.

The administration of an activated protein C or protein C like compound may be to subjects having one or more improved response polymorphism(s) in their protein C gene sequence. The administration of an activated protein C or protein C like compound may also be preferentially not made to subjects not having one or more improved response polymorphism(s) in their protein C gene.

An improved response polymorphism may be selected from one or more of the following: rs2069915GG; rs2069915AA; rs2069910CC; rs2069910TT; rs2069916CC; rs2069916TT; rs2069924CC; rs2069924TT; rs2069931 CC; rs2069931 TT; rs1799808CC; rs1799808TT; rs2069920CC; rs2069920TT; rs6714364AA; and rs6714364TT.

The activated protein C or protein C like compound may be Drotrecogin alfa activated.

### BRIEF DESCRIPTION OF THE DRAWINGS

FIG. 1. shows a change in mortality by genotype for Placebo and Xigris™-treated individuals, where all subjects have severe sepsis.

FIG. 2. shows a plot of the change in mortality by genotype for PROC rs2069915 in Caucasian individuals.

FIG. 3. shows a plot of the change in mortality by genotype for PROC rs2069915 in all subjects with severe sepsis and Apache II ≧25.

FIG. 4. shows a plot of the change in mortality by genotype for PROC rs2069915 in Caucasian subjects with severe sepsis and Apache II ≧25.

### DETAILED DESCRIPTION OF THE INVENTION

### 1. Definitions

In the description that follows, a number of terms are used extensively, the following definitions are provided to facilitate understanding of the invention.

“Activated protein C” or “protein C-like compound” as used herein includes any protein C molecule, protein C derivative, protein C variant, protein C analog and any pro-drug thereof, metabolite thereof, isomer thereof, combination of isomers thereof, or pharmaceutical composition of any of the preceding. Activated protein C or protein C-like compound or protein C-like compounds may be synthesized or purified. For example, Drotrecogin alfa (activated) is sold as XIGRIS™ by Eli Lilly and Company and has the same amino acid sequence as human plasma-derived Activated Protein C. Examples of derivatives, variants, analogs, or compositions etc. may be found in US patent applications: 20050176083; 20050143283; 20050095668; 20050059132; 20040028670; 20030207435; 20030027299; 20030022354; and 20030018175 and issued U.S. Pat. Nos. 6,933,367; 6,841,371; 6,815,533; 6,630,138; 6,630,137; 6,436,397; 6,395,270; 6,162,629; 6,159,468; 5,837,843; 5,453,373; 5,330,907; 5,766,921; 5,753,224; 5,516,650; and 5,358,932.

“Genetic material” includes any nucleic acid and can be a deoxyribonucleotide or ribonucleotide polymer in either single or double-stranded form.

“Nucleotides” are generally a purine (R) or pyrimidine (Y) base covalently linked to a pentose, usually ribose or deoxyribose, where the sugar carries one or more phosphate groups. Nucleic acids are generally a polymer of nucleotides joined by 3′-5′ phosphodiester linkages. As used herein “purine” is used to refer to the purine bases, A and G, and more broadly to include the nucleotide monomers, deoxyadenosine-5′-phosphate and deoxyguanosine-5′-phosphate, as components of a polynucleotide chain.

A “purine” is a heterocyclic organic compound containing fused pyrimidine and imidazole rings, and acts as the parent compound for purine bases, adenine (A) and guanine (G).

A “pyrimidine” is a single-ringed, organic base that forms nucleotide bases, cytosine (C), thymine (T) and uracil (U). As used herein “pyrimidine” is used to refer to the pyrimidine bases, C, T and U, and more broadly to include the pyrimidine nucleotide monomers that along with purine nucleotides are the components of a polynucleotide chain.

A nucleotide represented by the symbol M may be either an A or C, a nucleotide represented by the symbol W may be either an T/U or A, a nucleotide represented by the symbol Y may be either an C or T/U, a nucleotide represented by the symbol S may be either an G or C, while a nucleotide represented by the symbol R may be either an G or A, and a nucleotide represented by the symbol K may be either an G or T/U. Similarly, a nucleotide represented by the symbol V may be either A or G or C, while a nucleotide represented by the symbol D may be either A or G or T, while a nucleotide represented by the symbol B may be either G or C or T, and a nucleotide represented by the symbol H may be either A or C or T. Furthermore, a deletion or an insertion may be represented by either a “−” or “del” and “+” or “ins” or “I” respectively.

A “polymorphic site” or “polymorphism site” or “polymorphism” or “single nucleotide polymorphism site” (SNP site) or single nucleotide polymorphism” (SNP) as used herein is the locus or position within a given sequence at which divergence occurs. A “Polymorphism” is the occurrence of two or more forms of a gene or position within a gene (allele), in a population, in such frequencies that the presence of the rarest of the forms cannot be explained by mutation alone. The implication is that polymorphic alleles confer some selective advantage on the host. Preferred polymorphic sites have at least two alleles, each occurring at frequency of greater than 1%, and more preferably greater than 10% or 20% of a selected population. Polymorphic sites may be at known positions within a nucleic acid sequence or may be determined to exist using the methods described herein. Polymorphisms may occur in both the coding regions and the noncoding regions (for example, promoters, enhancers and introns) of genes. Polymorphisms may occur at a single nucleotide site (SNPs) or may involve an insertion or deletion as described herein.

An “improved response polymorphism” as used herein refers to an allelic variant or genotype at one or more polymorphic sites within the PROC gene as described herein, which predicts a subject will have a better response (for example, rs2069915AA or rs2069915GG) when activated protein C or protein C-like compound is administered (which may be measured by decreased mortality) as compared to an allelic variant or genotype or polymorphism (for example, rs2069915AG) which predicts a subject will respond less to activated protein C or protein C-like compound administration. A reduced response may be measured by a relative increase in mortality relative to subjects having an “improved response polymorphism”.

SNPs as described herein may also be associated with either a “predisposition or susceptibility to major organ dysfunction” or a “predisposition or susceptibility to an inflammatory condition” (for example organ failure or developing SIRS, sepsis or septic shock). Accordingly, certain SNPs may be assayed to determine whether a subject possesses a SNP allele that is indicative of an increased likelihood of experiencing major organ dysfunction or of developing an inflammatory condition. Whereas, other SNPs alleles may be assayed to determine whether a subject possesses a SNP allele that is indicative of a decreased likelihood of experiencing major organ dysfunction or of developing an inflammatory condition. SNP alleles that are associated with a decreased risk of experiencing major organ dysfunction or of developing an inflammatory condition may be referred to as “protective alleles”. Similarly, SNP alleles that are associated with an increased risk of experiencing major organ dysfunction or of developing an inflammatory condition may be referred to as “susceptibility alleles” or “risk alleles”.

In general, the detection of nucleic acids in a sample depends on the technique of specific nucleic acid hybridization in which the oligonucleotide is annealed under conditions of “high stringency” to nucleic acids in the sample, and the successfully annealed oligonucleotides are subsequently detected (see for example Spiegelman, S., Scientific American, Vol. 210, p. 48 (1964)). Hybridization under high stringency conditions primarily depends on the method used for hybridization, the oligonucleotide length, base composition and position of mismatches (if any). High stringency hybridization is relied upon for the success of numerous techniques routinely performed by molecular biologists, such as high stringency PCR, DNA sequencing, single strand conformational polymorphism analysis, and in situ hybridization. In contrast to Northern and Southern hybridizations, these techniques are usually performed with relatively short probes (e.g., usually about 16 nucleotides or longer for PCR or sequencing and about 40 nucleotides or longer for in situ hybridization). The high stringency conditions used in these techniques are well known to those skilled in the art of molecular biology, and examples of them can be found, for example, in AUSUBEL et al., Current Protocols in Molecular Biology, John Wiley & Sons, New York, N.Y., 1998.

“Oligonucleotides” as used herein are variable length nucleic acids or polynucleotides. Such oligonucleotides may be useful as probes, primers and in the manufacture of microarrays (arrays) for the detection and/or amplification of specific nucleic acids. Such DNA or RNA strands may be synthesized by the sequential addition (5′-3′ or 3′-5′) of activated monomers to a growing chain, which may be linked to an insoluble support. Numerous methods are known in the art for synthesizing oligonucleotides for subsequent individual use or as a part of the insoluble support, for example in arrays (BERNFIELD M R. and ROTTMAN F M. J. Biol. Chem. (1967) 242(18):4134-43; SULSTON J. et al. PNAS (1968) 60(2):409-415; GILLAM S. et al. Nucleic Acid Res. (1975) 2(5):613-624; BONORA G M. et al. Nucleic Acid Res. (1990) 18(11):3155-9; LASHKARI D A. et al. PNAS (1995) 92(17):7912-5; MCGALL G. et al. PNAS (1996) 93(24):13555-60; ALBERT T J. et al. Nucleic Acid Res. (2003) 31(7):e35; GAO X. et al. Biopolymers (2004) 73(5):579-96; and MOORCROFT M J. et al. Nucleic Acid Res. (2005) 33(8):e75). In general, oligonucleotides are synthesized through the stepwise addition of activated and protected monomers under a variety of conditions depending on the method being used. Subsequently, specific protecting groups may be removed to allow for further elongation and subsequently and once synthesis is complete all the protecting groups may be removed and the oligonucleotides removed from their solid supports for purification of the complete chains if so desired.

“Peptide nucleic acids” (PNA) as used herein refer to modified nucleic acids in which the sugar phosphate skeleton of a nucleic acid has been converted to an N-(2-aminoethyl)-glycine skeleton. Although the sugar-phosphate skeletons of DNA/RNA are subjected to a negative charge under neutral conditions resulting in electrostatic repulsion between complementary chains, the backbone structure of PNA does not inherently have a charge. Therefore, there is no electrostatic repulsion. Consequently, PNA has a higher ability to form double strands as compared with conventional nucleic acids, and has a high ability to recognize base sequences. Furthermore, PNAs are generally more robust than nucleic acids. PNAs may also be used in arrays and in other hybridization or other reactions as described above and herein for oligonucleotides.

An “array” of oligonucleotides as used herein refers to one or more oligonucleotides or peptide nucleic acids immobilized on a solid substrate, wherein discrete positions on the array are complementary to one or more of the provided polymorphic sequences. The oligonucleotides or peptide nucleic acids may be coupled directly or indirectly to a solid substrate. Coupling may be via chemical conjugated or through a linker molecule. Solid substrates may include, but are not limited to, beads (for example polystyrene beads), chips (for example: glass, plastic and silicon), plastic surfaces (for example: polystyrene and polycarbonate plastic multi-well plates) etc.

An “addressable collection” as used herein is a combination of nucleic acid molecules or peptide nucleic acids capable of being detected by, for example, the use of hybridization techniques or by any other means of detection known to those of ordinary skill in the art. A DNA microarray would be considered an example of an “addressable collection”.

In general the term “linkage”, as used in population genetics, refers to the co-inheritance of two or more non-allelic genes or sequences due to the close proximity of the loci on the same chromosome, whereby after meiosis they remain associated more often than the 50% expected for unlinked genes. However, during meiosis, a physical crossing (i.e. crossing-over) between individual chromatids may result in recombination.

“Recombination” generally occurs between large segments of DNA, whereby contiguous stretches of DNA and genes are likely to be moved together in the recombination event. Conversely, regions of the DNA that are far apart on a given chromosome are more likely to become separated during the process of crossing-over than regions of the DNA that are close together. Polymorphic molecular markers, like single nucleotide polymorphisms (SNPs), are often useful in tracking meiotic recombination events as positional markers on chromosomes.

Furthermore, the preferential occurrence of a disease gene in association with specific alleles of linked markers, such as SNPs or other polymorphisms, is called “Linkage Disequilibrium” (LD). This sort of disequilibrium generally implies that most of the disease chromosomes carry the same mutation and the markers being tested are relatively close to the disease gene(s).

For example, in SNP-based association analysis and linkage disequilibrium mapping, SNPs can be useful in association studies for identifying polymorphisms, associated with a pathological condition, such as sepsis. Unlike linkage studies, association studies may be conducted within the general population and are not limited to studies performed on related individuals in affected families. In a SNP association study the frequency of a given allele (i.e. SNP allele) is determined in numerous subjects having the condition of interest and in an appropriate control group. Significant associations between particular SNPs or SNP haplotypes and phenotypic characteristics may then be determined by numerous statistical methods known in the art.

Association analysis can either be direct or LD based. In direct association analysis, potentially causative SNPs may be tested as candidates for the pathogenic sequence. In LD based SNP association analysis, SNPs may be chosen at random over a large genomic region or even genome wide, to be tested for SNPs in LD with a pathogenic sequence or pathogenic SNP. Alternatively, candidate sequences associated with a condition of interest may be targeted for SNP identification and association analysis. Such candidate sequences usually are implicated in the pathogenesis of the condition of interest. In identifying SNPs associated with inflammatory conditions, candidate sequences may be selected from those already implicated in the pathway of the condition or disease of interest. Once identified, SNPs found in or associated with such sequences, may then be tested for statistical association with an individual's prognosis or susceptibility to the condition.

For an LD-based association analysis, high density SNP maps are useful in positioning random SNPs relative to an unknown pathogenic locus. Furthermore, SNPs tend to occur with great frequency and are often spaced uniformly throughout the genome. Accordingly, SNPs as compared with other types of polymorphisms are more likely to be found in close proximity to a genetic locus of interest. SNPs are also mutationally more stable than variable number tandem repeats (VNTRs).

In population genetics LD refers to the “preferential association of a particular allele, for example, a mutant allele for a disease with a specific allele at a nearby locus more frequently than expected by chance” and implies that alleles at separate loci are inherited as a single unit (GELEHRTER, T. D., COLLINS, F. S. (1990). Principles of Medical Genetics. Baltimore: Williams & Wilkens). Accordingly, the alleles at these loci and the haplotypes constructed from their various combinations serve as useful markers of phenotypic variation due to their ability to mark clinically relevant variability at a particular position, such as position 301 of SEQ ID NO:1. (eg. AKEY J et al. (2001) Eur J HumGenet. 9:291-300; and ZHANG, K. et al. (2002) Am J Hum Genet. 71:1386-1394). This viewpoint is further substantiated by KHOURY et al. ((1993). Fundamentals of Genetic Epidemiology New York: Oxford University Press at p. 160) who state, “[w]henever the marker allele is closely linked to the true susceptibility allele and is in [linkage] disequilibrium with it, one can consider that the marker allele can serve as a proxy for the underlying susceptibility allele.”

As used herein LD is the occurrence in a population of certain combinations of linked alleles in greater proportion than expected from the allele frequencies at the loci. For example, the preferential occurrence of a disease gene in association with specific alleles of linked markers, such as SNPs, or between specific alleles of linked markers, are considered to be in LD. This sort of disequilibrium generally implies that most of the disease chromosomes carry the same mutation and that the markers being tested are relatively close to the disease gene(s). Accordingly, if the genotype of a first locus is in LD with a second locus (or third locus etc.), the determination of the allele at only one locus would necessarily provide the identity of the allele at the other locus. When evaluating loci for LD those sites within a given population having a high degree of LD (i.e. an absolute value for D′ of ≧0.5 or r²≧0.5) are potentially useful in predicting the identity of an allele of interest (i.e. associated with the condition of interest). A high degree of LD may be represented by an absolute value for D′ of ≧0.6 or r²≧0.6. Alternatively, a high degree of LD may be represented by an absolute value for D′ of ≧0.7 or r²≧0.7 or by an absolute value for D′ of ≧0.8 or r²≧0.8. Additionally, a high degree of LD may be represented by an absolute value for D′ of ≧0.85 or r²≧0.85 or by an absolute value for D′ of ≧0.9 or r²≧0.9. Accordingly, two SNPs that have a high degree of LD may be equally useful in determining the identity of the allele of interest or disease allele. Therefore, we may assume that knowing the identity of the allele at one SNP may be representative of the allele identity at another SNP in LD. Accordingly, the determination of the genotype of a single locus can provide the identity of the genotype of any locus in LD therewith and the higher the degree of LD the more likely that two SNPs may be used interchangeably. For example, in the population from which the tagged SNPs were identified from the SNP identified by rs2069915 is in LD with the SNP identified by rs2069910, whereby when the genotype of rs2069915 is A the genotype of rs2069910 is T. Similarly, when the genotype of rs2069915 is G the genotype of rs2069910 is C. Accordingly, the determination of the genotype at rs2069915 will provide the identity of the genotype at rs2069910 or any other locus in LD therewith. Particularly, where such a locus is has a high degree of LD thereto.

LD is useful for genotype-phenotype association studies. For example, if a specific allele at one SNP site (e.g. “A”) is the cause of a specific clinical outcome (e.g. “B”) in a genetic association study then, by mathematical inference, any SNP (e.g. “C”) which is in significant LD with the first SNP, will show some degree of association with the clinical outcome. That is, if A is associated (˜) with B, i.e. A˜B and C˜A then it follows that C˜SB. Of course, the SNP that will be most closely associated with the specific clinical outcome, B, is the causal SNP—the genetic variation that is mechanistically responsible for the clinical outcome. Thus, the degree of association between any SNP, C, and clinical outcome will depend on LD between A and C.

Until the mechanism underlying the genetic contribution to a specific clinical outcome is fully understood, LD helps identify potential candidate SNPs and also helps identify a range of SNPs that may be clinically useful for prognosis of clinical outcome or of treatment effect. If one SNP within a gene is found to be associated with a specific clinical outcome, then other SNPs in LD will also have some degree of association and therefore some degree of prognostic usefulness. By way of prophetic example, if multiple polymorphisms were tested for individual association with an improved response to activated protein C or protein C-like compound administration in our sepsis cohort of ICU patients, wherein the multiple polymorphisms had a range of LD with PROC polymorphism rs2069915 and it was assumed that rs2069915 was the causal polymorphism, and we were to order the polymorphisms by the degree of LD with rs2069915, we would expect to find that polymorphisms with high degrees of LD with rs2069915 would also have a high degree of association with this specific clinical outcome. As LD decreased, we would expect the degree of association of the polymorphism with an improved response to activated protein C or protein C-like compound administration to also decrease. Accordingly, logic dictates that if A˜B and C˜A, then C˜B. That is, any polymorphism, whether already discovered or as yet undiscovered, that is in LD with one of the improved response polymorphisms described herein will likely be a predictor of the same clinical outcomes that rs2069915 is a predictor of. The similarity in prediction between this known or unknown polymorphism and rs2069915 would depend on the degree of LD between such a polymorphism and rs2069915.

A site has been identified as a polymorphic site in the PROC gene (see TABLE 1A). Furthermore, the rs2069915 polymorphism in TABLE 1A is linked to (in LD with) numerous polymorphisms as set out in TABLE 1B below and may also therefore be indicative of subject prognosis.

TABLE 1A
Polymorphisms in PROC genotyped in a cohort of critically ill patients
with severe sepsis. Minor allele frequencies are from European data
annotated in dbSNP (NCBI).

| rs Identifier | Chromosomal Position | Alleles | Minor Allele |
|---|---|---|---|
| (rsID) | (NCBI build 36) | Observed | Frequency |
| 2069915 | 127894848 | G/A | 0.348 |

TABLE 1B
Polymorphisms in LD with the PROC SNP listed in TABLE 1A above, as
identified using the LD-select algorithm (CARLSON CS. et al. Am. J.
Hum. Genet. (2004) 74: 106-120), r² ≧ 0.5/minor allele frequency
(MAF) = 0.05.

| | | | | Chromosomal |
|---|---|---|---|---|
| | Observed | LD SNP | LD | position (NCBI |
| SNP rsID | Alleles | rsID | Alleles | Build 36) |
| rs2069915 | G/A | n/a | n/a | 127894848 |
| | | rs2069910 | C/T | 127894444 |
| | | rs2069916 | C/T | 127895885 |
| | | rs2069924 | C/T | 127899464 |
| | | rs2069931 | C/T | 127902039 |
| | | rs1799808 | C/T | 127892332 |
| | | rs2069920 | C/T | 127896116 |
| | | rs6714364 | A/T | 127899427 |

It will be appreciated by a person of skill in the art that the numerical designations of the positions of polymorphisms within a sequence are relative to the specific sequence. Also the same positions may be assigned different numerical designations depending on the way in which the sequence is numbered and the sequence chosen, as illustrated by the alternative numbering of the equivalent polymorphisms in the scientific literature. Furthermore, sequence variations within the population, such as insertions or deletions, may change the relative position and subsequently the numerical designations of particular nucleotides at and around a polymorphic site.

Polymorphic sites in SEQ ID NO:1 and SEQ ID NO:2-8 are identified by their variant designation (i.e. M, W, Y, S, R, K, V, B, D, H or by “−” for a deletion, a “+” or “G” etc. for an insertion).

An “rs” prefix designates a SNP in the database is found at the NCBI SNP database (http://www.ncbi.nlm.nih.gov/entrez/query.fcgi?db=Snp). The “rs” numbers are the NCBI|rsSNP ID form.

TABLE 1C below shows the flanking sequences for protein C gene SNPs providing the rs designations, alleles and corresponding SEQ ID NO designations. The polymorphisms are identified in bold and underlined text, and the position is given in the left-hand column.

TABLE 1C
PROC SNPs and respective flanking sequences

| SNP rs ID | SEQ ID: | FLANKING SEQUENCE |
|---|---|---|
| rs2069915 | 1 | GGCTGTTGGTTTCATTTGTGCCTTTATAGA |
| (position | | GCTGTTTATCTGCTTGGGACCTGCACCTCC |
| 301) | | ACCCTTTCCCAAGGTGCCCTCAGCTCAGGC |
| | | ATACCCTCCTCTAGGATGCCTTTTCCCCCA |
| | | TCCCTTCTTGCTCACACCCCCAACTTGATC |
| | | TCTCCCTCCTAACTGTGCCCTGCACCCAAG |
| | | ACAGACACTTCACAGAGCCCAGGAGACACC |
| | | TGGGGACCCTTCCTGGGTGATAGGTCTGTC |
| | | TATCCTCCAGGTGTCCCTGCCCAAGGGGAG |
| | | AAGCATGGGGAATACTTGGTTGGGGGAGGA |
| | | RAGGAAGACTGGGGGGATGTGTCAAGATGG |
| | | GGCTGCACGTGGTGTACTGGCAGAAGAGTG |
| | | AGAGGATTTAACTTGGCAGCCTTTACAGCA |
| | | GCAGCCAGGGCTTGAGTACTTATCTCTGGG |
| | | CCAGGGACTGTATTGGATGTTTTACATGAC |
| | | GGTCTCATCCCCATGTTTTTGGATGAGTAA |
| | | ATTGAACCTTAGAAAGGTAAAGACACTGGC |
| | | TCAAGGTCACACAGAGATCGGGGTGGGGTT |
| | | CACAGGGAGGCCTGTCCATCTCAGAGCAAG |
| | | GCTTCGTCCTCCAACTGCCATCTGCTTCCT |
| | | G |

The Sequence given in TABLE 1C (SEQ ID NO:1) above and in TABLE 1D (SEQ ID NOS:2-8) would be useful to a person of skill in the art in the design of primers and probes or other oligonucleotides for the identification of protein C gene SNP alleles and or genotypes as described herein.

TABLE 1D
Flanking sequences of PROC SNPs in
LD with rs2069915

| | SEQ | |
|---|---|---|
| | ID | |
| SNP | NO: | FLANKING SEQUENCE |
| rs2069910 | 2 | CCATCTCTCTGAGCCCTGGGTGAGGTGAGGGG |
| (at position | | CAGATGGGAATGGCAGGAATCAACTGACAAGT |
| 301) | | CCCAGGTAGGCCAGCTGCCAGAGTGCCACACA |
| | | GGGGCTGCCAGGGCAGGCATGCGTGATGGCAG |
| | | GGAGCCCCGCGATGACCTCCTAAAGCTCCCTC |
| | | CTCCACACGGGGATGGTCACAGAGTCCCCTGG |
| | | GCCTTCCCTCTCCACCCACTCACTCCCTCAAC |
| | | TGTGAAGACCCCAGGCCCAGGCTACCGTCCAC |
| | | ACTATCCAGCACAGCCTCCCCTACTCAAATGC |
| | | ACACTGGCCTCAYGGCTGCCCTGCCCCAACCC |
| | | CTTTCCTGGTCTCCACAGCCAACGGGAGGAGG |
| | | CCATGATTCTTGGGGAGGTCCGCAGGACACAT |
| | | GGGCCCCTAAAGCCACACCAGGCTGTTGGTTT |
| | | CATTTGTGCCTTTATAGAGCTGTTTATCTGCT |
| | | TGGGACCTGCACCTCCACCCTTTCCCAAGGTG |
| | | CCCTCAGCTCAGGCATACCCTCCTCTAGGATG |
| | | CCTTTTCCCCCATCCCTTCTTGCTCACACCCC |
| | | CAACTTGATCTCTCCCTCCTAACTGTGCCCTG |
| | | CACCCAAGACAGACACTTCACAGAG |
| rs2069916 | 3 | ATCTGCTTGGGACCTGCACCTCCACCCTTTCC |
| (at position | | CAAGGTGCCCTCAGCTCAGGCATACCCTCCTC |
| 301) | | TAGGATGCCTTTTCCCCCATCCCTTCTTGCTC |
| | | ACACCCCCAACTTGATCTCTCCCTCCTAACTG |
| | | TGCCCTGCACCCAAGACAGACACTTCACAGAG |
| | | CCCAGGAGACACCTGGGGACCCTTCCTGGGTG |
| | | ATAGGTCTGTCTATCCTCCAGGTGTCCCTGCC |
| | | CAAGGGGAGAAGCATGGGGAATACTTGGTTGG |
| | | GGGAGGAGAGGAAGACTGGGGGGATGTGTCAA |
| | | GATGGGGCTGCAYGTGGTGTACTGGCAGAAGA |
| | | GTGAGAGGATTTAACTTGGCAGCCTTTACAGC |
| | | AGCAGCCAGGGCTTGAGTACTTATCTCTGGGC |
| | | CAGGGACTGTATTGGATGTTTTACATGACGGT |
| | | CTCATCCCCATGTTTTTGGATGAGTAAATTGA |
| | | ACCTTAGAAAGGTAAAGACACTGGCTCAAGGT |
| | | CACACAGAGATCGGGGTGGGGTTCACAGGGAG |
| | | GCCTGTCCATCTCAGAGCAAGGCTTCGTCCTC |
| | | CAACTGCCATCTGCTTCCTGGGGAGGAAAAGA |
| | | GCAGAGGACCCCTGCGCCAAGCCAT |
| rs2069924 | 4 | AACTCTCAACATGCCCTCCCACCTGCACTGCC |
| (at position | | TTCCTGGAAGCCCCACAGCCTCCTATGGTTCC |
| 301) | | GTGGTCCAGTCCTTCAGCTTCTGGGCGCCCCC |
| | | ATCACGGGCTGAGATTTTTGCTTTCCAGTCTG |
| | | CCAAGTCAGTTACTGTGTCCATCCATCTGCTG |
| | | TCAGCTTCTGGAATTGTTGCTGTTGTGCCCTT |
| | | TCCATTCTTTTGTTATGATGCAGCTCCCCTGC |
| | | TGACGACGTCCCATTGCTCTTTTAAGTCTAGA |
| | | TATCTGGACTGGGCATTCAAGGCCCATTTTGA |
| | | GCAGAGTCGGGCYGACCTTTCAGCCCTCAGTT |
| | | CTCCATGGAGTATGCGCTCTCTTCTTGGCAGG |
| | | GAGGCCTCACAAACATGCCATGCCTATTGTAG |
| | | GAGCTCTCCAAGAATGCTCACCTCCTTCTCCC |
| | | TGTAATTCCTTTCCTCTGTGAGGAGACTCAGC |
| | | AGCTCCCATTATGAGACCTTACTAATCCCAGG |
| | | GATCACCCCCAACAGCCCTGGGGTACAATGAG |
| | | CTTTTAAGAAGTTTAACCACCTATGTAAGGAG |
| | | ACACAGGCAGTGGGCGATGCTGCCTGGCCTGA |
| | | CTCTTGCCATTGGGTGGTACTGTTT |
| rs2069931 | 5 | CGATCTATGGCAATTTCTGGAGGGGGGGTCTG |
| (at position | | GCTCAACTCTTTATGCCAAAAAGAAGGCAAAG |
| 301) | | CATATTGAGAAAGGCCAAATTCACATTTCCTA |
| | | CAGCATAATCTATGGCCAGTGGCCCCCCGTGG |
| | | GGCTTGGCTTAGAATTCCCAGGTGCTCTTCCC |
| | | AGGGAACCATCAGTCTGGACTGAGAGGACCTT |
| | | CTCTCTCAGGTGGGACCCGGCCCTGTCCTCCC |
| | | TGGCAGTGCCGTGTTCTGGGGGTCCTCCTCTC |
| | | TGGGTCTCACTGCCCCTGGGGTCTCTCCAGCT |
| | | ACCTTTGCTCCAYGTTCCTTTGTGGCTCTGGT |
| | | CTGTGTCTGGGGTTTCCAGGGGTCTCGGGCTT |
| | | CCCTGCTGCCCATTCCTTCTCTGGTCTCACGG |
| | | CTCCGTGACTCCTGAAAACCAACCAGCATCCT |
| | | ACCTCTTTGGGATTGACACCTGTTGGCCACTC |
| | | CTTCTGGCAGGAAAAGTCACCGTTGATAGGGT |
| | | TCCACGGCATAGACAGGTGGCTCCGCGCCAGT |
| | | GCCTGGGACGTGTGGGTGCACAGTCTCCGGGT |
| | | GAACCTTCTTCAGGCCCTCTGCCCAGGCCTGC |
| | | AGGGGCACAGCAGTGGGTGGGCCTC |
| rs1799808 | 6 | AAAAATAGAGGTTAGAGGGATGCTATGTGCCA |
| (at position | | TTGTGTGTGTGTGTTGGGGGTGGGGATTGGGG |
| 433) | | GTGATTTGTGAGCAATTGGAGGTGAGGGTGGA |
| | | GCCCAGTGCCCAGCACCTATGCACTGGGGACC |
| | | CAAAAAGGAGCATCTTCTCATGATTTTATGTA |
| | | TCAGAAATTGGGATGGCATGTCATTGGGACAG |
| | | CGTCTTTTTTCTTGTATGGTGGCACATAAATA |
| | | CATGTGTCTTATAATTAATGGTATTTTAGATT |
| | | TGACGAAATATGGAATATTACCTGTTGTGCTG |
| | | ATCTTGGGCAAACTATAATATCTCTGGGCAAA |
| | | AATGTCCCCATCTGAAAAACAGGGACAACGTT |
| | | CCTCCCTCAGCCAGCCACTATGGGGCTAAAAT |
| | | GAGACCACATCTGTCAAGGGTTTTGCCCTCAC |
| | | CTCCCTCCCTGCTGGAYGGCATCCTTGGTGGG |
| | | CAGAGGTGGGCTTCGGGCAGAACAAGCCGTGC |
| | | TGAGCTAGGACCAGGAGTGCTAGTGCCACTGT |
| | | TTGTCTATGGAGAGGGAGGCCTCAGTGCTGAG |
| | | GGCCAAGCAAATATTTGTGGTTATGGATTAAC |
| | | TCGAACTCCAGGCTGTCATGGCGGCAGGACGG |
| | | CGAACTTGCAGTATCTCCACGACCC |
| rs2069920 | 7 | TCACATCAAAGGGAGAAAATCTGATTGTTCAG |
| (at position | | GGGGTCGGAAGACAGGGTCTGTGTCCTATTTG |
| 201) | | TCTAAGGGTCAGAGTCCTTTGGAGCCCCCAGA |
| | | GTCCTGTGGACGTGGCCCTAGGTAGTAGGGTG |
| | | AGCTTGGTAACGGGGCTGGCTTCCTGAGACAA |
| | | GGCTCAGACCCGCTCTGTCCCTGGGGATCGCT |
| | | TCAGCCACYAGGACCTGAAAATTGTGCACGCC |
| | | TGGGCCCCCTTCCAAGGCATCCAGGGATGCTT |
| | | TCCAGTGGAGGCTTTCAGGGCAGGAGACCCTC |
| | | TGGCCTGCACCCTCTCTTGCCCTCAGCCTCCA |
| | | CCTCCTTGACTGGACCCCCATCTGGACCTCCA |
| | | TCCCCACCACCTCTTTCCCCAGTGGCCTCCCT |
| | | GGCAGACACCACAGTGA |
| rs6714364 | 8 | CTTCTGCAGGCAGGGAGAGGGGAGTCAAGTCA |
| (at position | | GTGAGGAGGGCTTTCGCAGTTTCTCTTACAAA |
| 326) | | CTCTCAACATGCCCTCCCACCTGCACTGCCTT |
| | | CCTGGAAGCCCCACAGCCTCCTATGGTTCCGT |
| | | GGTCCAGTCCTTCAGCTTCTGGGCGCCCCCAT |
| | | CACGGGCTGAGATTTTTGCTTTCCAGTCTGCC |
| | | AAGTCAGTTACTGTGTCCATCCATCTGCTGTC |
| | | AGCTTCTGGAATTGTTGCTGTTGTGCCCTTTC |
| | | CATTCTTTTGTTATGATGCAGCTCCCCTGCTG |
| | | ACGACGTCCCATTGCTCTTTTAAGTCTAGATA |
| | | TCTGGWCTGGGCATTCAAGGCCCATTTTGAGC |
| | | AGAGTCGGGCCGACCTTTCAGCCCTCAGTTCT |
| | | CCATGGAGTATGCGCTCTCTTCTTGGCAGGGA |
| | | GGCCTCACAAACATGCCATGCCTATTGTAGGA |
| | | GCTCTCCAAGAATGCTCACCTCCTTCTCCCTG |
| | | TAATTCCTTTCCTCTGTGAGGAGCTCAGCAGC |
| | | ATCCCATTATGAGA |

TABLE 1D shows the flanking sequences for PROC SNPs in LD with rs2069915 in TABLE 1C.
Each SNP is identified in bold and underlined text, and the position is given in the left-hand column.

An “allele” is defined as any one or more alternative forms of a given gene. In a diploid cell or organism the members of an allelic pair (i.e. the two alleles of a given gene) occupy corresponding positions (loci) on a pair of homologous chromosomes and if these alleles are genetically identical the cell or organism is said to be “homozygous”, but if genetically different the cell or organism is said to be “heterozygous” with respect to the particular gene.

A “gene” is an ordered sequence of nucleotides located in a particular position on a particular chromosome that encodes a specific functional product and may include untranslated and untranscribed sequences in proximity to the coding regions (5′ and 3′ to the coding sequence). Such non-coding sequences may contain regulatory sequences needed for transcription and translation of the sequence or introns etc. or may as yet to have any function attributed to them beyond the occurrence of the SNP of interest.

A “genotype” is defined as the genetic constitution of an organism, usually in respect to one gene or a few genes or a region of a gene relevant to a particular context (i.e. the genetic loci responsible for a particular phenotype).

A “phenotype” is defined as the observable characters of an organism.

TABLE 1E shows a genotype correlation for a protein C gene SNP with values representing an indication of responsiveness to treatment of an inflammatory condition with activated protein C or protein C-like compound.

TABLE 1E
rs2069915 genotypes and predicted response to treatment with activated
protein C or protein C-like compound

| | | Responsiveness To |
|---|---|---|
| SNP | Genotype | Treatment^{∞} |
| rs2069915 | AA | R |
| rs2069915 | GA | NR |
| rs2069915 | GG | R |

R = Responsive
NR = Nonresponsive

A “single nucleotide polymorphism” (SNP) occurs at a polymorphic site occupied by a single nucleotide, which is the site of variation between allelic sequences. The site is usually preceded by and followed by highly conserved sequences of the allele (e.g., sequences that vary in less than 1/100 or 1/1000 members of the populations). A single nucleotide polymorphism usually arises due to substitution of one nucleotide for another at the polymorphic site. A “transition” is the replacement of one purine by another purine or one pyrimidine by another pyrimidine. A “transversion” is the replacement of a purine by a pyrimidine or vice versa. Single nucleotide polymorphisms can also arise from a deletion (represented by “−” or “del”) of a nucleotide or an insertion (represented by “+” or “ins” or “I”) of a nucleotide relative to a reference allele. Furthermore, a person of skill in the art would appreciate that an insertion or deletion within a given sequence could alter the relative position and therefore the position number of another polymorphism within the sequence. Furthermore, although an insertion or deletion may by some definitions not qualify as a SNP as it may involve the deletion of or insertion of more than a single nucleotide at a given position, as used herein such polymorphisms are also called SNPs as they generally result from an insertion or deletion at a single site within a given sequence.

A “systemic inflammatory response syndrome” or (SIRS) is defined as including both septic (i.e. sepsis or septic shock) and non-septic systemic inflammatory response (i.e. post operative). “SIRS” is further defined according to ACCP (American College of Chest Physicians) guidelines as the presence of two or more of A) temperature >38° C. or <36° C., B) heart rate >90 beats per minute, C) respiratory rate >20 breaths per minute, and D) white blood cell count >12,000 per mm³ or <4,000 mm³. In the following description, the presence of two, three, or four of the “SIRS” criteria were scored each day over the 28 day observation period.

“Sepsis” is defined as the presence of at least two “SIRS” criteria and known or suspected source of infection. Septic shock was defined as sepsis plus one new organ failure by Brussels criteria plus need for vasopressor medication.

Subject outcome or prognosis as used herein refers the ability of a subject to recover from an inflammatory condition and may be used to determine the efficacy of a treatment regimen, for example the administration of activated protein C or protein C-like compound. An inflammatory condition, may be selected from the group consisting of: sepsis, septicemia, pneumonia, septic shock, systemic inflammatory response syndrome (SIRS), Acute Respiratory Distress Syndrome (ARDS), acute lung injury, aspiration pneumanitis, infection, pancreatitis, bacteremia, peritonitis, abdominal abscess, inflammation due to trauma, inflammation due to surgery, chronic inflammatory disease, ischemia, ischemia-reperfusion injury of an organ or tissue, tissue damage due to disease, tissue damage due to chemotherapy or radiotherapy, and reactions to ingested, inhaled, infused, injected, or delivered substances, glomerulonephritis, bowel infection, opportunistic infections, and for subjects undergoing major surgery or dialysis, subjects who are immunocompromised, subjects on immunosuppressive agents, subjects with HIV/AIDS, subjects with suspected endocarditis, subjects with fever, subjects with fever of unknown origin, subjects with cystic fibrosis, subjects with diabetes mellitus, subjects with chronic renal failure, subjects with acute renal failure, oliguria, subjects with acute renal dysfunction, glomerulo-nephritis, interstitial-nephritis, acute tubular necrosis (ATN), subjects with bronchiectasis, subjects with chronic obstructive lung disease, chronic bronchitis, emphysema, or asthma, subjects with febrile neutropenia, subjects with meningitis, subjects with septic arthritis, subjects with urinary tract infection, subjects with necrotizing fasciitis, subjects with other suspected Group A streptococcus infection, subjects who have had a splenectomy, subjects with recurrent or suspected enterococcus infection, other medical and surgical conditions associated with increased risk of infection, Gram positive sepsis, Gram negative sepsis, culture negative sepsis, fungal sepsis, meningococcemia, post-pump syndrome, cardiac stun syndrome, myocardial infarction, stroke, congestive heart failure, hepatitis, epiglotittis, E. coli 0157:H7, malaria, gas gangrene, toxic shock syndrome, pre-eclampsia, eclampsia, HELP syndrome, mycobacterial tuberculosis, Pneumocystic carinii, pneumonia, Leishmaniasis, hemolytic uremic syndrome/thrombotic thrombocytopenic purpura, Dengue hemorrhagic fever, pelvic inflammatory disease, Legionella, Lyme disease, Influenza A, Epstein-Barr virus, encephalitis, inflammatory diseases and autoimmunity including Rheumatoid arthritis, osteoarthritis, progressive systemic sclerosis, systemic lupus erythematosus, inflammatory bowel disease, idiopathic pulmonary fibrosis, sarcoidosis, hypersensitivity pneumonitis, systemic vasculitis, Wegener's granulomatosis, transplants including heart, liver, lung kidney bone marrow, graft-versus-host disease, transplant rejection, sickle cell anemia, nephrotic syndrome, toxicity of agents such as OKT3, cytokine therapy, cirrhosis, disseminated intravascular coagulation (DIC), cardiogenic shock, and acute kidney injury.

Assessing subject outcome, prognosis, or response of a subject to activated protein C or protein C-like compound administration may be accomplished by various methods. For Example, an “APACHE II” score is defined as Acute Physiology And Chronic Health Evaluation and herein was calculated on a daily basis from raw clinical and laboratory variables. Vincent et al. (VINCENT J L. FERREIRA F. MORENO R. (2000) Crit. Care Clin. 16:353-366) summarize APACHE score as follows “First developed in 1981 by Knaus et al., the APACHE score has become the most commonly used survival prediction model in ICUs worldwide. The APACHE II score, a revised and simplified version of the original prototype, uses a point score based on initial values of 12 routine physiologic measures, age, and previous health status to provide a general measure of severity of disease. The values recorded are the worst values taken during the subject's first 24 hours in the ICU. The score is applied to one of 34 admission diagnoses to estimate a disease-specific probability of mortality (APACHE II predicted risk of death). The maximum possible APACHE II score is 71, and high scores have been well correlated with mortality. The APACHE II score has been widely used to stratify and compare various groups of critically ill subjects, including subjects with sepsis, by severity of illness on entry into clinical trials.” Furthermore, the criteria or indication for administering activated vasopressin (XIGRIS™-Drotrecogin alfa (activated)) in the United States is an APACHE II score of >25. In Europe, the criteria or indication for administering activated protein C or protein C-like compound is an APACHE II score of ≧25 or 2 new organ system failures.

“Critically ill” as used herein is meant to include subjects that have an APACHE II score of ≧25 or 2 new organ system failures.

“Activated protein C” as used herein includes Drotrecogin alfa (activated) which is sold as XIGRIS™ by Eli Lilly and Company. Drotrecogin alfa (activated) is a serine protease glycoprotein of approximately 55 kilodalton molecular weight and having the same amino acid sequence as human plasma-derived Activated Protein C. The protein consists of a heavy chain and a light chain linked by a disulfide bond. XIGRIS™, Drotrecogin alfa (activated) is currently indicated for the reduction of mortality in adult subjects with severe sepsis (sepsis associated with acute organ dysfunction) who have a high risk of death (e.g., as determined by an APACHE II score of greater >25 or having 2 or more organ system failures).

XIGRIS™ is available in 5 mg and 20 mg single-use vials containing sterile, preservative-free, lyophilized drug. The vials contain 5.3 mg and 20.8 mg of Drotrecogin alfa (activated), respectively. The 5 and 20 mg vials of XIGRIS™ also contain 40.3 and 158.1 mg of sodium chloride, 10.9 and 42.9 mg of sodium citrate, and 31.8 and 124.9 mg of sucrose, respectively. XIGRIS™ is recommended for intravenous administration at an infusion rate of 24 mcg/kg/hr for a total duration of infusion of 96 hours. Dose adjustment based on clinical or laboratory parameters is not recommended. If the infusion is interrupted, it is recommended that when restarted the infusion rate should be 24 mcg/kg/hr. Dose escalation or bolus doses of Drotrecogin alfa are not recommended. XIGRIS™ may be reconstituted with Sterile Water for Injection and further diluted with sterile normal saline injection. These solutions must be handled so as to minimize agitation of the solution (Product information. XIGRIS™, Drotrecogin alfa (activated), Eli Lilly and Company, November 2001).

Drotrecogin alfa (activated) is a recombinant form of human Activated Protein C, which may be produced using a human cell line expressing the complementary DNA for the inactive human Protein C zymogen, whereby the cells secrete protein into the fermentation medium. The protein may be enzymatically activated by cleavage with thrombin and subsequently purified. Methods, DNA compounds and vectors for producing recombinant activated human protein C are described in U.S. Pat. Nos. 4,775,624; 4,992,373; 5,196,322; 5,270,040; 5,270,178; 5,550,036; 5,618,714 all of which are incorporated herein by reference.

Treatment of sepsis using activated protein C or protein C-like compound in combination with a bactericidal and endotoxin neutralizing agent is described in U.S. Pat. No. 6,436,397;methods for processing protein C is described in U.S. Pat. No. 6,162,629; protein C derivatives are described in U.S. Pat. Nos. 5,453,373 and 6,630,138; glycosylation mutants are described in U.S. Pat. No. 5,460,953; and Protein C formulations are described in U.S. Pat. Nos. 6,630,137, 6,436,397, 6,395,270 and 6,159,468, all of which are incorporated herein by reference.

A “Brussels score” score is a method for evaluating organ dysfunction as compared to a baseline. If the Brussels score is 0 (i.e. moderate, severe, or extreme), then organ failure was recorded as present on that particular day (see TABLE 2A below). In the following description, to correct for deaths during the observation period, days alive and free of organ failure (DAF) were calculated as previously described. For example, acute lung injury was calculated as follows. Acute lung injury is defined as present when a subject meets all of these four criteria. 1) Need for mechanical ventilation, 2) Bilateral pulmonary infiltrates on chest X-ray consistent with acute lung injury, 3) PaO₂/FiO₂ ratio is less than 300, 4) No clinical evidence of congestive heart failure or if a pulmonary artery catheter is in place for clinical purposes, a pulmonary capillary wedge pressure less than 18 mm Hg (1). The severity of acute lung injury is assessed by measuring days alive and free of acute lung injury over a 28 day observation period. Acute lung injury is recorded as present on each day that the person has moderate, severe or extreme dysfunction as defined in the Brussels score. Days alive and free of acute lung injury is calculated as the number of days after onset of acute lung injury that a subject is alive and free of acute lung injury over a defined observation period (28 days). Thus, a lower score for days alive and free of acute lung injury indicates more severe acute lung injury. The reason that days alive and free of acute lung injury is preferable to simply presence or absence of acute lung injury, is that acute lung injury has a high acute mortality and early death (within 28 days) precludes calculation of the presence or absence of acute lung injury in dead subjects. The cardiovascular, renal, neurologic, hepatic and coagulation dysfunction were similarly defined as present on each day that the person had moderate, severe or extreme dysfunction as defined by the Brussels score. Days alive and free of steroids are days that a person is alive and is not being treated with exogenous corticosteroids (e.g. hydrocortisone, prednisone, methylprednisolone). Days alive and free of pressors are days that a person is alive and not being treated with intravenous vasopressors (e.g. dopamine, norepinephrine, epinephrine, phenylephrine). Days alive and free of an International Normalized Ratio (INR) >1.5 are days that a person is alive and does not have an INR >1.5.

TABLE 2A
Brussels Organ Dysfunction Scoring System

| | Free of Organ | | Clinically Significant | | |
|---|---|---|---|---|---|
| | Dysfunction | | Organ Dysfunction | | |
| | Normal | Mild | Moderate | Severe | Extreme |
| DAF ORGAN | 1 | | 0 | | |
| DYSFUNCTION | | | | | |
| SCORE | | | | | |
| Cardiovascular | >90 | ≦90 | ≦90 | ≦90 plus | ≦90 plus |
| Systolic BP | | Responsive to | Unresponsive to | pH ≦ 7.3 | pH ≦ 7.2 |
| (mmHg) | | fluid | fluid | | |
| Pulmonary | >400 | 400-301 | 300-201 | 200-101 | ≦100 |
| Pₐo₂/F_{I}o₂ (mmHg) | | | Acute lung | ARDS | Severe ARDS |
| | | | injury | | |
| Renal | <1.5 | 1.5-1.9 | 2.0-3.4 | 3.5-4.9 | ≧5.0 |
| Creatinine (mg/Dl) | | | | | |
| Hepatic | <1.2 | 1.2-1.9 | 2.0-5.9 | 6.0-11.9 | ≧12 |
| Bilirubin (mg/dL) | | | | | |
| Hematologic | >120 | 120-81 | 80-51 | 50-21 | ≦20 |
| Platelets | | | | | |
| (×10⁵/mm³) | | | | | |
| Neurologic | 15 | 14-13 | 12-10 | 9-6 | ≦5 |
| (Glascow Score) | | | | | |

Round Table Conference on Clinical Trials for the Treatment of Sepsis Brussels, Mar. 12-14, 1994.

Analysis of variance (ANOVA) is a standard statistical approach to test for significant differences between sets of measurements.

### General Methods

One aspect of the invention may involve the identification of subjects or the selection of subjects that are either at risk of developing and inflammatory condition or the identification of subjects who already have an inflammatory condition. For example, subjects who have undergone major surgery or scheduled for or contemplating major surgery may be considered as being at risk of developing an inflammatory condition. Furthermore, subjects may be determined as having an inflammatory condition using diagnostic methods and clinical evaluations known in the medical arts. An inflammatory condition, may be selected from the group consisting of: sepsis, septicemia, pneumonia, septic shock, systemic inflammatory response syndrome (SIRS), Acute Respiratory Distress Syndrome (ARDS), acute lung injury, aspiration pneumanitis, infection, pancreatitis, bacteremia, peritonitis, abdominal abscess, inflammation due to trauma, inflammation due to surgery, chronic inflammatory disease, ischemia, ischemia-reperfusion injury of an organ or tissue, tissue damage due to disease, tissue damage due to chemotherapy or radiotherapy, and reactions to ingested, inhaled, infused, injected, or delivered substances, glomerulonephritis, bowel infection, opportunistic infections, and for subjects undergoing major surgery or dialysis, subjects who are immunocompromised, subjects on immunosuppressive agents, subjects with HIV/AIDS, subjects with suspected endocarditis, subjects with fever, subjects with fever of unknown origin, subjects with cystic fibrosis, subjects with diabetes mellitus, subjects with chronic renal failure, subjects with acute renal failure, oliguria, subjects with acute renal dysfunction, glomerulo-nephritis, interstitial-nephritis, acute tubular necrosis (ATN), subjects with bronchiectasis, subjects with chronic obstructive lung disease, chronic bronchitis, emphysema, or asthma, subjects with febrile neutropenia, subjects with meningitis, subjects with septic arthritis, subjects with urinary tract infection, subjects with necrotizing fasciitis, subjects with other suspected Group A streptococcus infection, subjects who have had a splenectomy, subjects with recurrent or suspected enterococcus infection, other medical and surgical conditions associated with increased risk of infection, Gram positive sepsis, Gram negative sepsis, culture negative sepsis, fungal sepsis, meningococcemia, post-pump syndrome, cardiac stun syndrome, myocardial infarction, stroke, congestive heart failure, hepatitis, epiglotittis, E. coli 0157:H7, malaria, gas gangrene, toxic shock syndrome, pre-eclampsia, eclampsia, HELP syndrome, mycobacterial tuberculosis, Pneumocystic carinii, pneumonia, Leishmaniasis, hemolytic uremic syndrome/thrombotic thrombocytopenic purpura, Dengue hemorrhagic fever, pelvic inflammatory disease, Legionella, Lyme disease, Influenza A, Epstein-Barr virus, encephalitis, inflammatory diseases and autoimmunity including Rheumatoid arthritis, osteoarthritis, progressive systemic sclerosis, systemic lupus erythematosus, inflammatory bowel disease, idiopathic pulmonary fibrosis, sarcoidosis, hypersensitivity pneumonitis, systemic vasculitis, Wegener's granulomatosis, transplants including heart, liver, lung kidney bone marrow, graft-versus-host disease, transplant rejection, sickle cell anemia, nephrotic syndrome, toxicity of agents such as OKT3, cytokine therapy, cirrhosis, disseminated intravascular coagulation (DIC), cardiogenic shock, and acute kidney injury.

Once a subject is identified as being at risk for developing or having an inflammatory condition or is to be administered activated protein C, then genetic sequence information may be obtained from the subject. Or alternatively genetic sequence information may already have been obtained from the subject. For example, a subject may have already provided a biological sample for other purposes or may have even had their genetic sequence determined in whole or in part and stored for future use. Genetic sequence information may be obtained in numerous different ways and may involve the collection of a biological sample that contains genetic material. Particularly, genetic material, containing the sequence or sequences of interest. Many methods are known in the art for collecting bodily samples and extracting genetic material from those samples. Genetic material can be extracted from blood, tissue and hair and other samples. There are many known methods for the separate isolation of DNA and RNA from biological material. Typically, DNA may be isolated from a biological sample when first the sample is lysed and then the DNA is isolated from the lysate according to any one of a variety of multi-step protocols, which can take varying lengths of time. DNA isolation methods may involve the use of phenol (Sambrook, J. et al., “Molecular Cloning”, Vol. 2, pp. 9.14-9.23, Cold Spring Harbor Laboratory Press (1989) and Ausubel, Frederick M. et al., “Current Protocols in Molecular Biology”, Vol. 1, pp. 2.2.1-2.4.5, John Wiley & Sons, Inc. (1994)). Typically, a biological sample is lysed in a detergent solution and the protein component of the lysate is digested with proteinase for 12-18 hours. Next, the lysate is extracted with phenol to remove most of the cellular components, and the remaining aqueous phase is processed further to isolate DNA. In another method, described in Van Ness et al. (U.S. Pat. No. 5,130,423), non-corrosive phenol derivatives are used for the isolation of nucleic acids. The resulting preparation is a mix of RNA and DNA.

Other methods for DNA isolation utilize non-corrosive chaotropic agents. These methods, which are based on the use of guanidine salts, urea and sodium iodide, involve lysis of a biological sample in a chaotropic aqueous solution and subsequent precipitation of the crude DNA fraction with a lower alcohol. The final purification of the precipitated, crude DNA fraction can be achieved by any one of several methods, including column chromatography (Analects, (1994) Vol 22, No. 4, Pharmacia Biotech), or exposure of the crude DNA to a polyanion-containing protein as described in Koller (U.S. Pat. No. 5,128,247).

Yet another method of DNA isolation, which is described by Botwell, D. D. L. (Anal. Biochem. (1987) 162:463-465) involves lysing cells in 6M guanidine hydrochloride, precipitating DNA from the lysate at acid pH by adding 2.5 volumes of ethanol, and washing the DNA with ethanol.

Numerous other methods are known in the art to isolate both RNA and DNA, such as the one described by CHOMCZYNSKI (U.S. Pat. No. 5,945,515), whereby genetic material can be extracted efficiently in as little as twenty minutes. EVANS and HUGH (U.S. Pat. No. 5,989,431) describe methods for isolating DNA using a hollow membrane filter.

Once a subject's genetic material has been obtained from the subject it may then be further be amplified by Reverse Transcription Polymerase Chain Reaction (RT-PCR), Polymerase Chain Reaction (PCR), Transcription Mediated Amplification (TMA), Ligase chain reaction (LCR), Nucleic Acid Sequence Based Amplification (NASBA) or other methods known in the art, and then further analyzed to detect or determine the presence or absence of one or more polymorphisms or mutations in the sequence of interest, provided that the genetic material obtained contains the sequence of interest. Particularly, a person may be interested in determining the presence or absence of a mutation in the protein C gene sequence, as described in herein. The sequence of interest may also include other mutations, or may also contain some of the sequence surrounding the mutation of interest.

Detection or determination of a nucleotide identity, or the presence of one or more single nucleotide polymorphism(s) (SNP typing), may be accomplished by any one of a number methods or assays known in the art. Many DNA typing methodologies are useful detection of SNPs. The majority of SNP genotyping reactions or assays can be assigned to one of four broad groups (sequence-specific hybridization, primer extension, oligonucleotide ligation and invasive cleavage). Furthermore, there are numerous methods for analyzing/detecting the products of each type of reaction (for example, fluorescence, luminescence, mass measurement, electrophoresis, etc.). Furthermore, reactions can occur in solution or on a solid support such as a glass slide, a chip, a bead, etc.

In general, sequence-specific hybridization involves a hybridization probe, which is capable of distinguishing between two DNA targets differing at one nucleotide position by hybridization. Usually probes are designed with the polymorphic base in a central position in the probe sequence, whereby under optimized assay conditions only the perfectly matched probe target hybrids are stable and hybrids with a one base mismatch are unstable. A strategy which couples detection and sequence discrimination is the use of a “molecular beacon”, whereby the hybridization probe (molecular beacon) has 3′ and 5′ reporter and quencher molecules and 3′ and 5′ sequences which are complementary such that absent an adequate binding target for the intervening sequence the probe will form a hairpin loop. The hairpin loop keeps the reporter and quencher in close proximity resulting in quenching of the fluorophor (reporter) which reduces fluorescence emissions. However, when the molecular beacon hybridizes to the target the fluorophor and the quencher are sufficiently separated to allow fluorescence to be emitted from the fluorophor.

Similarly, primer extension reactions (i.e. mini sequencing, nucleotide-specific extensions, or simple PCR amplification) are useful in sequence discrimination reactions. For example, in mini sequencing a primer anneals to its target DNA immediately upstream of the SNP and is extended with a single nucleotide complementary to the polymorphic site. Where the nucleotide is not complementary, no extension occurs.

Oligonucleotide ligation assays require two sequence-specific probes and one common ligation probe per SNP. The common ligation probe hybridizes adjacent to a sequence-specific probe and when there is a perfect match of the appropriate sequence-specific probe, the ligase joins both the sequence-specific and the common probes. Where there is not a perfect match the ligase is unable to join the sequence-specific and common probes. Probes used in hybridization can include double-stranded DNA, single-stranded DNA and RNA oligonucleotides, and peptide nucleic acids. Hybridization methods for the identification of single nucleotide polymorphisms or other mutations involving a few nucleotides are described in the U.S. Pat. Nos. 6,270,961; 6,025,136; and 6,872,530. Suitable hybridization probes for use in accordance with the invention include oligonucleotides and PNAs from about 10 to about 400 nucleotides, alternatively from about 20 to about 200 nucleotides, or from about 30 to about 100 nucleotides in length.

Alternatively, an invasive cleavage method requires an oligonucleotide called an Invader™ probe and sequence-specific probes to anneal to the target DNA with an overlap of one nucleotide. When the sequence-specific probe is complementary to the polymorphic base, overlaps of the 3′ end of the invader oligonucleotide form a structure that is recognized and cleaved by a Flap endonuclease releasing the 5′ arm of the allele specific probe.

5′ exonuclease activity or TaqMan™ assay (Applied Biosystems™) is based on the 5′ nuclease activity of Taq polymerase that displaces and cleaves the oligonucleotide probes hybridized to the target DNA generating a fluorescent signal. It is necessary to have two probes that differ at the polymorphic site wherein one probe is complementary to the ‘normal’ sequence and the other to the mutation of interest. These probes have different fluorescent dyes attached to the 5′ end and a quencher attached to the 3′ end when the probes are intact the quencher interacts with the fluorophor by fluorescence resonance energy transfer (FRET) to quench the fluorescence of the probe. During the PCR annealing step the hybridization probes hybridize to target DNA. In the extension step the 5′ fluorescent dye is cleaved by the 5′ nuclease activity of Taq polymerase, leading to an increase in fluorescence of the reporter dye. Mismatched probes are displaced without fragmentation. The presence of a mutation in a sample is determined by measuring the signal intensity of the two different dyes.

It will be appreciated that numerous other methods for sequence discrimination and detection are known in the art and some of which are described in further detail below. It will also be appreciated that reactions such as arrayed primer extension mini sequencing, tag microarrays and sequence-specific extension could be performed on a microarray. One such array based genotyping platform is the microsphere based tag-it high throughput genotyping array (BORTOLIN S. et al. Clinical Chemistry (2004) 50(11): 2028-36). This method amplifies genomic DNA by PCR followed by sequence-specific primer extension with universally tagged genotyping primers. The products are then sorted on a Tag-It array and detected using the Luminex xMAP™ system.

Mutation detection methods may include but are not limited to the following:

Restriction Fragment Length Polymorphism (RFLP) strategy—An RFLP gel-based analysis can be used to indicate the presence or absence of a specific mutation at polymorphic sites within a gene. Briefly, a short segment of DNA (typically several hundred base pairs) is amplified by PCR. Where possible, a specific restriction endonuclease is chosen that cuts the short DNA segment when one polymorphism is present but does not cut the short DNA segment when the polymorphism is not present, or vice versa. After incubation of the PCR amplified DNA with this restriction endonuclease, the reaction products are then separated using gel electrophoresis. Thus, when the gel is examined the appearance of two lower molecular weight bands (lower molecular weight molecules travel farther down the gel during electrophoresis) indicates that the DNA sample had a polymorphism was present that permitted cleavage by the specific restriction endonuclease. In contrast, if only one higher molecular weight band is observed (at the molecular weight of the PCR product) then the initial DNA sample had the polymorphism that could not be cleaved by the chosen restriction endonuclease. Finally, if both the higher molecular weight band and the two lower molecular weight bands are visible then the DNA sample contained both polymorphisms, and therefore the DNA sample, and by extension the subject providing the DNA sample, was heterozygous for this polymorphism;

Sequencing—For example the Maxam-Gilbert technique for sequencing (MAXAM A M. and GILBERT W. Proc. Natl. Acad. Sci. USA (1977) 74(4):560-564) involves the specific chemical cleavage of terminally labeled DNA. In this technique four samples of the same labeled DNA are each subjected to a different chemical reaction to effect preferential cleavage of the DNA molecule at one or two nucleotides of a specific base identity. The conditions are adjusted to obtain only partial cleavage, DNA fragments are thus generated in each sample whose lengths are dependent upon the position within the DNA base sequence of the nucleotide(s) which are subject to such cleavage. After partial cleavage is performed, each sample contains DNA fragments of different lengths, each of which ends with the same one or two of the four nucleotides. In particular, in one sample each fragment ends with a C, in another sample each fragment ends with a C or a T, in a third sample each ends with a G, and in a fourth sample each ends with an A or a G. When the products of these four reactions are resolved by size, by electrophoresis on a polyacrylamide gel, the DNA sequence can be read from the pattern of radioactive bands. This technique permits the sequencing of at least 100 bases from the point of labeling. Another method is the dideoxy method of sequencing was published by SANGER et al., (Proc. Natl. Acad. Sci. USA (1977) 74(12):5463-5467). The Sanger method relies on enzymatic activity of a DNA polymerase to synthesize sequence-dependent fragments of various lengths. The lengths of the fragments are determined by the random incorporation of dideoxynucleotide base-specific terminators. These fragments can then be separated in a gel as in the Maxam-Gilbert procedure, visualized, and the sequence determined. Numerous improvements have been made to refine the above methods and to automate the sequencing procedures. Similarly, RNA sequencing methods are also known. For example, reverse transcriptase with dideoxynucleotides have been used to sequence encephalomyocarditis virus RNA (ZIMMERN D. and KAESBERG P. Proc. Natl. Acad. Sci. USA (1978) 75(9):4257-4261). MILLS D R. and KRAMER F R. (Proc. Natl. Acad. Sci. USA (1979) 76(5):2232-2235) describe the use of Q-beta replicase and the nucleotide analog inosine for sequencing RNA in a chain-termination mechanism. Direct chemical methods for sequencing RNA are also known (PEATTIE D A. Proc. Natl. Acad. Sci. USA (1979) 76(4):1760-1764). Other methods include those of Donis-Keller et al. (1977, Nucl. Acids Res. 4:2527-2538), SIMONCSITS A. et al. (Nature (1977) 269(5631):833-836), AXELROD V D. et al. (Nucl. Acids Res.(1978) 5(10):3549-3563), and KRAMER F R. and MILLS D R. (Proc. Natl. Acad. Sci. USA (1978) 75(11):5334-5338). Nucleic acid sequences can also be read by stimulating the natural fluoresce of a cleaved nucleotide with a laser while the single nucleotide is contained in a fluorescence enhancing matrix (U.S. Pat. No. 5,674,743); In a mini sequencing reaction, a primer that anneals to target DNA adjacent to a SNP is extended by DNA polymerase with a single nucleotide that is complementary to the polymorphic site. This method is based on the high accuracy of nucleotide incorporation by DNA polymerases. There are different technologies for analyzing the primer extension products. For example, the use of labeled or unlabeled nucleotides, ddNTP combined with dNTP or only ddNTP in the mini sequencing reaction depends on the method chosen for detecting the products;

Probes used in hybridization may include double-stranded DNA, single-stranded DNA and RNA oligonucleotides, and peptide nucleic acids. Hybridization methods for the identification of single nucleotide polymorphisms or other mutations involving a few nucleotides are described in the U.S. Pat. Nos. 6,270,961; 6,025,136; and 6,872,530. Suitable hybridization probes for use in accordance with the invention include oligonucleotides and PNAs from about 10 to about 400 nucleotides, alternatively from about 20 to about 200 nucleotides, or from about 30 to about 100 nucleotides in length.

A template-directed dye-terminator incorporation with fluorescent polarization-detection (TDI-FP) method is described by FREEMAN B D. et al. (J Mol Diagnostics (2002) 4(4):209-215) for large scale screening;

Oligonucleotide ligation assay (OLA) is based on ligation of probe and detector oligonucleotides annealed to a polymerase chain reaction amplicon strand with detection by an enzyme immunoassay (VILLAHERMOSA M L. J Hum Virol (2001) 4(5):238-48; ROMPPANEN E L. Scand J Clin Lab Invest (2001) 61(2):123-9; IANNONE M A. et al. Cytometry (2000) 39(2):131-40);

Ligation-Rolling Circle Amplification (L-RCA) has also been successfully used for genotyping single nucleotide polymorphisms as described in QI X. et al. Nucleic Acids Res (2001) 29(22):E116;

5′ nuclease assay has also been successfully used for genotyping single nucleotide polymorphisms (AYDIN A. et al. Biotechniques (2001) (4):920-2, 924, 926-8.);

Polymerase proofreading methods are used to determine SNPs identities, as described in WO 0181631;

Detection of single base pair DNA mutations by enzyme-amplified electronic transduction is described in PATOLSKY F et al. Nat. Biotech. (2001) 19(3):253-257;

Gene chip technologies are also known for single nucleotide polymorphism discrimination whereby numerous polymorphisms may be tested for simultaneously on a single array (EP 1120646 and GILLES P N. et al. Nat. Biotechnology (1999) 17(4):365-70);

Matrix assisted laser desorption ionization time of flight (MALDI-TOF) mass spectroscopy is also useful in the genotyping single nucleotide polymorphisms through the analysis of microsequencing products (HAFF L A. and SMIRNOV I P. Nucleic Acids Res. (1997) 25(18):3749-50; HAFF L A. and SMIRNOV I P. Genome Res. (1997) 7:378-388; SUN X. et al. Nucleic Acids Res. (2000) 28 e68; BRAUN A. et al. Clin. Chem. (1997) 43:1151-1158; LITTLE D P. et al. Eur. J. Clin. Chem. Clin. Biochem. (1997) 35:545-548; FEI Z. et al. Nucleic Acids Res. (2000) 26:2827-2828; and BLONDAL T. et al. Nucleic Acids Res. (2003) 31(24):e155).

Sequence-specific PCR methods have also been successfully used for genotyping single nucleotide polymorphisms (HAWKINS J R. et al. Hum Mutat (2002) 19(5):543-553). Alternatively, a Single-Stranded Conformational Polymorphism (SSCP) assay or a Cleavase Fragment Length Polymorphism (CFLP) assay may be used to detect mutations as described herein.

Alternatively, if a subject's sequence data is already known, then obtaining may involve retrieval of the subjects nucleic acid sequence data (for example from a database), followed by determining or detecting the identity of a nucleic acid or genotype at a polymorphic site by reading the subject's nucleic acid sequence at the one or more polymorphic sites.

Once the identity of a polymorphism(s) is determined or detected an indication may be obtained as to subject response to activated protein C or protein C like compound or protein C like compound administration based on the genotype (the nucleotide at the position) of the polymorphism of interest. As described herein, polymorphisms in protein C gene sequences, may be used to predict a subject's response to activated protein C or protein C like compound treatment. Methods for predicting a subject's response to activated protein C or protein C like compound treatment may be useful in making decisions regarding the administration of activated protein C.

Methods of treatment of an inflammatory condition in a subject having an improved response polymorphism in the protein C pathway gene are described herein. An improved response may include an improvement subsequent to administration of said therapeutic agent, whereby the subject has an increased likelihood of survival, reduced likelihood of organ damage or organ dysfunction (Brussels score), an improved APACHE II score, days alive and free of pressors, inotropes, and reduced systemic dysfunction (cardiovascular, respiratory, ventilation, CNS, coagulation [INR>1.5], renal and/or hepatic).

As described above genetic sequence information or genotype information may be obtained from a subject wherein the sequence information contains one or more polymorphic sites in the protein C gene sequence. Also, as previously described the sequence identity of one or more polymorphisms in the protein C gene sequence of one or more subjects may then be detected or determined. Furthermore, subject response to administration of activated protein C or protein C like compound may be assessed as described above. For example, the APACHE II scoring system or the Brussels score may be used to assess a subject's response to treatment by comparing subject scores before and after treatment. Once subject response has been assessed, subject response may be correlated with the sequence identity of one or more polymorphism(s). The correlation of subject response may further include statistical analysis of subject outcome scores and polymorphism(s) for a number of subjects.

### Methods

Study Design and Phenotype Data Collection This study was conducted as a double-blind, randomized, placebo-controlled multicentre trial (BERNARD G R et al. (2001) N Engl J Med 344(10):699-709). Study subjects with severe sepsis were enrolled and randomized to receive either placebo or XIGRIS™. Severe sepsis was defined as having a known or suspected infection at the time of screening, having at least three SIRS criteria and at least one new organ dysfunction. Baseline characteristics including demographic variables, preexisting conditions, organ dysfunction, disease severity and laboratory indices were evaluated within the 24 hours before the start of the infusion. Patient samples were anonymized using a one-way encryption process that effectively strips personal identifiers from the record. The study endpoint and primary outcome variable was defined prospectively as death from any cause and assessed for 28 days after the start of therapy. Days alive and free (DAF) of organ dysfunction were defined as secondary outcome variables and are outlined in Table 3. During each 24-hour period, DAF was scored for each organ dysfunction measure with a score of 1 being assigned if the patient was alive and free of organ dysfunction. A score of 0 was assigned if the patient developed organ dysfunction or died during that 24-hour period.

TABLE 3
Organ dysfunction variables

| Organ Dysfunction | Measure |
|---|---|
| Coagulation | Overt DIC at baseline (as defined by |
| | ISTH criteria) |
| Mechanical Ventilation | Ventilated with p02/Fi02 <300 at baseline |
| Mild Renal Dysfunction | Creatinine <2 mg/dL at baseline |
| Multiple Organ Dysfunction | Organ dysfunction ≧2 at baseline |

### Genotyping

DNA was extracted from blood spotted on Whatman™ FTA cards and genotyped for polymorphisms in PROC using the iPLEX™ platform (Sequenom™, San Diego, Calif.). Table 4 contains the NCBI “rs” identifier numbers (rs Id), the chromosomal position of each SNP genotyped and the alleles observed.

TABLE 4
chromosomal positions and observed alleles for SNPs genotyped in
this study

| | NCBI chromosomal | |
|---|---|---|
| rs Id | position (build 36) | Alleles observed |
| rs2069915 | 127894848 | G/A |

### Statistical Methods

Logistic regression was performed by rs2069915 genotype to test the following two null hypotheses using 28-day survival as the predictor variable:
a) PROC rs2069915 genotype does not predict 28-day survival
b) PROC rs2069915 genotype does not predict response to administration of Xigris™ as measured by 28-day survival.

Individuals were stratified by treatment (i.e. placebo-treated or Xigris™-treated) and logistic regression analysis was undertaken first for all study participants (n=1568) and then by the following subgroups:
1. Caucasians (n=1290)
2. Apache II >25 (n=752)
3. Caucasian and Apache II >25 (n=613)

Adjustments were made for baseline characteristics if they were observed to covary with the primary outcome variable of 28-day survival.

### Results

### Example 1

### rs2069915 Risk of Death and Xigris™ Response by Genotype: All Subjects with Severe Sepsis

Table 5 shows the distribution of genotypes for all subjects and all subjects stratified by treatment group. No difference in genotype is observed between placebo- and Xigris™-treated subjects (X²; p=0.15).

TABLE 5
rs2069915 genotype data for all study subjects with severe sepsis

| | rs 2069915 Genotypes | | |
|---|---|---|---|
| Group (n) | GG (n) | AG (n) | AA (n) |
| Total (1568) | 0.37 (575) | 0.45 (711) | 0.18 (282) |
| Placebo (788) | 0.39 (307) | 0.43 (341) | 0.18 (140) |
| Xigris ™ (780) | 0.34 (268) | 0.47 (370) | 0.18 (142) |

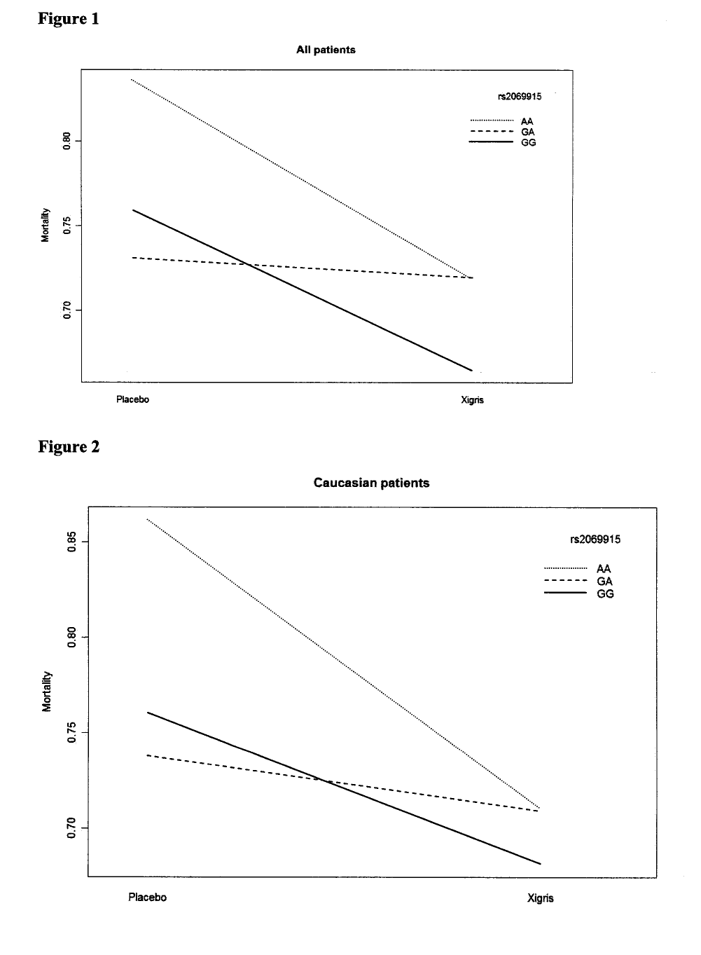

The difference in mortality by genotype between all placebo- and Xigris™-treated subjects with severe sepsis is illustrated in FIG. 1.

Table 6 shows the results from the logistic regression analysis under a categorical model by rs2069915 genotype and the interaction of rs2069915 genotype with Xigris™ treatment. In the absence of Xigris™ treatment, AG individuals are observed to have a significantly decreased risk of death compared to AA individuals (p=0.0147). A similar trend is observed for GG individuals who also have a decreased risk of mortality compared to those who are AA (p=0.0695). Evaluating the effects of Xigris™ treatment by genotype suggests that there is a trend towards decreased risk of mortality for AA individuals who are administered Xigris™ when compared with those AA individuals treated with placebo (p=0.0616).

TABLE 6
Logistic regression statistics for all subjects with severe sepsis

| rs2069915 | Genotype | | Treatment log | |
|---|---|---|---|---|
| Genotypes | log odds | p Genotype | odds | p Treatment |
| AG vs AA | −0.6310 | 0.0147 | 0.6340 | 0.0616 |
| GG vs AA | −0.4797 | 0.0695 | 0.2256 | 0.5173 |
| GG vs AG | 0.1513 | 0.4027 | −0.401 | 0.1031 |

### Example 2

### rs2069915 Risk of Death and Xigris™ Response by Genotype: All Caucasian Subjects

Table 7 shows the distribution of genotypes for all Caucasian subjects with severe sepsis and all Caucasians with severe sepsis stratified by treatment group. No difference in genotype by treatment group is observed (X²; p=0.51).

TABLE 7
rs2069915 genotype data for all Caucasian subjects with severe sepsis

| | rs 2069915 Genotypes | | |
|---|---|---|---|
| Group | GG (n) | AG (n) | AA (n) |
| Total (1290) | 0.36 (468) | 0.48 (614) | 0.16 (208) |
| Placebo (642) | 0.38 (242) | 0.46 (298) | 0.17 (108) |
| Xigris ™ (648) | 0.35 (226) | 0.49 (316) | 0.15 (100) |

The difference in mortality by genotype between placebo and Xigris™-treated Caucasian individuals is demonstrated in FIG. 2.

Table 8 shows the results from the logistic regression analysis under a categorical model by rs2069915 genotype and the interaction of rs2069915 genotype with Xigris™ treatment. In the absence of Xigris™ treatment, AG and GG individuals are observed to have a significantly decreased risk of death compared to AA individuals (p=0.0105 and 0.0342 respectively). However, when administered Xigris™, AA individuals are observed to have a significantly reduced risk of death (p=0.0496) as demonstrated by the difference in mortality observed between AA placebo- and AA Xigris™-treated individuals.

TABLE 8
Logistic regression statistics for all Caucasian individuals with severe
sepsis

| rs2069915 | Genotype | | Treatment log | |
|---|---|---|---|---|
| Genotypes | log odds | p Genotype | odds | p Treatment |
| AG vs AA | −0.7876 | 0.0105 | 0.7821 | 0.0496 |
| GG vs AA | −0.6701 | 0.0342 | 0.5350 | 0.1932 |
| GG vs AG | 0.1176 | 0.5568 | −0.8979 | 0.3692 |

### Example 3

### rs2069915 Risk of Death and Xigris™ Response by Genotype: All Subjects with Severe Sepsis and Apache II ≧25

Table 9 shows the distribution of genotypes for all subjects with severe sepsis and Apache II ≧25 and all subjects with Apache II ≧25 stratified by treatment group. No difference in genotype by treatment group is observed (X²; p=0.54).

TABLE 9
rs2069915 genotype data for Xigris ™-treated individuals with Apache
II ≧ 25

| | rs 2069915 Genotypes | | |
|---|---|---|---|
| Group (n) | GG (n) | AG (n) | AA (n) |
| Total (752) | 0.39 (290) | 0.43 (321) | 0.19 (141) |
| Placebo (382) | 0.40 (154) | 0.41 (156) | 0.19 (72) |
| Xigris ™ (370) | 0.37 (136) | 0.45 (165) | 0.19 (69) |

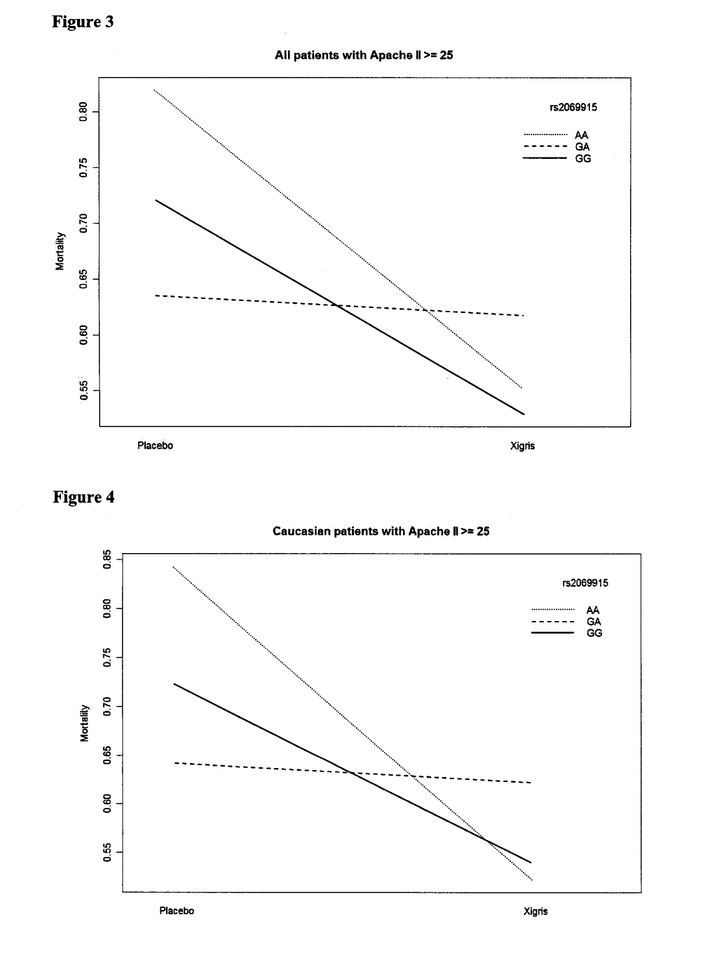

The difference in mortality by genotype between placebo- and Xigris™-treated subjects with severe sepsis and Apache II ≧25 is illustrated in FIG. 3.

Table 10 shows the results from the logistic regression analysis for all subjects with severe sepsis and Apache II ≧25 under a categorical model by rs2069915 genotype and the interaction of rs2069915 genotype with Xigris™ treatment. In the absence of Xigris™ treatment, AG individuals are observed to have a significantly decreased risk of death when compared to AA individuals (p=0.0059). However, when administered Xigris™, AA and GG individuals are observed to have a significantly reduced risk of death compared to AG individuals as demonstrated by the intra-genotype differences in mortality observed for AA and GG placebo- and Xigris™-treated individuals (p=0.0063 and p=0.0250 respectively).

TABLE 10
Logistic regression statistics by genotype and Xigris ™ treatment for all
individuals with severe sepsis and Apache II ≧ 25

| rs2069915 | genotype log | genotype p | treatment log | treatment p |
|---|---|---|---|---|
| genotypes | odds | value | odds | value |
| AG vs AA | −0.9605 | 0.0059 | 1.2388 | 0.0063 |
| GG vs AA | −0.5643 | 0.1121 | 0.4784 | 0.3013 |
| GG vs AG | 0.3962 | 0.1055 | −0.7603 | 0.0250 |

### Example 4

### rs2069915 Risk of Death and Xigris™ Response by Genotype: Caucasians with Severe Sepsis and Apache II >25

Table 11 shows the distribution of genotypes for all Caucasian subjects with Apache II ≧25 and all Caucasians with Apache II ≧25 stratified by treatment group. No difference in genotype by treatment group is observed (X²; p=0.71).

TABLE 11
raw genotype data for Caucasians with severe sepsis and Apache II ≧ 25

| | rs2069915 Genotypes | | |
|---|---|---|---|
| Group (n) | GG | AG | AA |
| Total (613) | 0.38 (236) | 0.46 (280) | 0.16 (97) |
| Placebo (382) | 0.32 (123) | 0.36 (137) | 0.13 (51) |
| Xigris ™ (370) | 0.31 (113) | 0.39 (143) | 0.12 (46) |

The difference in mortality by genotype between placebo- and Xigris™-treated Caucasian subjects with severe sepsis and Apache II ≧25 is illustrated in FIG. 4.

Table 12 shows the results from the logistic regression analysis for Caucasians with severe sepsis and Apache II ≧25 under a categorical model by rs2069915 genotype and the interaction of rs2069915 genotype with Xigris™ treatment. In the absence of Xigris™ treatment, AG individuals are observed to have a significantly decreased risk of death when compared to AA individuals (p=0.0098). A similar trend is observed between GG and AA individuals with GG subjects having decreased mortality compared to those who are AA (0.0978). However, when administered Xigris™, AA individuals are observed to have a significantly reduced risk of death compared to AG individuals as demonstrated by the intra-genotype differences in mortality observed for AA placebo- and Xigris™-treated individuals (p=0.0056). Similarly, GG Xigris™-treated individuals are observed to have a strong trend towards decreased mortality compared with those subjects treated with placebo.

TABLE 12
Logistic regression statistics by genotype and Xigris ™ treatment for
Caucasian subjects with severe sepsis and Apache II ≧ 25

| rs2069915 | genotype log | genotype p | treatment log | treatment p |
|---|---|---|---|---|
| genotype | odds | value | odds | value |
| AG vs AA | −1.0962 | 0.0098 | 1.5089 | 0.0056 |
| GG vs AA | −0.7195 | 0.0978 | 0.7921 | 0.1559 |
| GG vs AG | 0.3767 | 0.1615 | −0.7168 | 0.0534 |

Polymorphism rs2069915 (PROC gene) predicts decreased risk of mortality and improved response to Xigris™.

### Linkage Disequilibrium Analysis

Polymorphisms found to be in LD with the polymorphism identified in Table 1A as having a decreased risk of mortality and/or improved response to Xigris™ are listed in Table 1B. The polymorphisms in LD were identified using the LD-select algorithm which analyzes patterns of linkage disequilibrium between polymorphic SNPs across all gene regions of interest (CARLSON C S. et al. Am. J. Hum. Genet. (2004) 74:106-120), r²≧0.5/minor allele frequency (MAF)=0.05. The binning algorithm used in LD-select identified all SNPs that exceed the r² threshold of or >0.5 with our IRP SNPs. A minimum minor allele frequency of 0.05 was used throughout the analysis.

Although the foregoing invention has been described in some detail by way of illustration and example for purposes of clarity of understanding, it will be readily apparent to those of skill in the art in light of the teachings of this invention that changes and modification may be made thereto without departing from the spirit or scope of the appended claims.

1. A method for identifying a subject predisposed or susceptible to major organ dysfunction or predisposed or susceptible to sepsis, septic shock, or systemic inflammatory response syndrome (SIRS), the method comprising determining a genotype of said subject at one or more of polymorphic sites in the subject's protein C gene selected from: rs2069915 and one or more polymorphic sites in linkage disequilibrium thereto, selected from one or more of the following: rs2069910; rs2069916; rs2069924; rs2069931; rs1799808; rs2069920; and rs6714364, wherein the genotype is determined using a nucleic acid sample from the subject.

2. The method of claim 1, wherein the subject is critically ill.

3. The method of claim 1, further comprising obtaining protein C gene sequence information for the subject.

4. (canceled)

5. The method of claim 1, further comprising obtaining the nucleic acid sample from the subject.

6. The method of claim 1, further comprising administration of an activated protein C or protein C like compound, wherein the subject has one or more improved response polymorphism(s) in their protein C gene sequence selected from rs2069915 AA or rs2069915 GG; or one or more polymorphic sites in linkage disequilibrium thereto selected from the following: rs2069910 CC or rs2069910 TT; rs2069916 CC or rs2069916 TT; rs2069924 CC or rs2069924 TT; rs2069931 CC or rs2069931 TT; rs1799808 CC or rs1799808 TT; rs2069920 CC or rs2069920 TT; and rs671464 AA or rs671464 TT.

7. The method of claim 6, wherein a subject not having one or more improved response polymorphism(s) in their protein C gene sequences is selectively not administered activated protein C or protein C like compound.

8-13. (canceled)

14. The method of claim 1, wherein said genotype is determined using one or more of the following techniques:
(a) restriction fragment length analysis;
(b) sequencing;
(c) micro-sequencing assay;
(d) hybridization;
(e) invader assay;
(f) gene chip hybridization assays;
(g) oligonucleotide ligation assay;
(h) ligation rolling circle amplification;
(i) 5′ nuclease assay;
(j) polymerase proofreading methods;
(k) allele specific PCR;
(l) matrix assisted laser desorption ionization time of flight (MALDI-TOF) mass spectroscopy;
(m) ligase chain reaction assay;
(n) enzyme-amplified electronic transduction; and
(o) single base pair extension assay.

15-19. (canceled)

20. A method of selecting a subject for the treatment of sepsis, septic shock, or systemic inflammatory response syndrome (SIRS) with an activated protein C or protein C like compound, comprising the step of identifying a subject wherein the subject has an improved response polymorphism as follows: rs2069915 AA or rs2069915 GG; or one or more polymorphic sites in linkage disequilibrium thereto selected from the following: rs2069910 CC or rs2069910 TT; rs2069916 CC or rs2069916 TT; rs2069924 CC or rs2069924 TT; rs2069931 CC or rs2069931 TT; rs1799808 CC or rs1799808 TT; rs2069920 CC or rs2069920 TT; and rs671464 AA or rs671464 TT, wherein the identification of a subject with the improved response polymorphism is predictive of increased responsiveness to the treatment of the inflammatory condition with the activated protein C or protein C like compound.

21-22. (canceled)

23. The method of claim 20, further comprising determining the subject's APACHE II score as an assessment of subject risk.

24. The method of claim 20, further comprising determining the number of organ system failures for the subject as an assessment of subject risk.

25. The method of claim 23, wherein the subject's APACHE II score is indicative of an increased risk when ≧25.

26. The method of claim 24, wherein 2 or more organ system failures are indicative of increased subject risk.

27-29. (canceled)

30. The method claim 20, wherein the activated protein C or protein C like compound is Drotrecogin alfa activated.

31. A composition for determining the identity of two or more polymorphisms selected from the group consisting of rs2069915; rs2069910; rs2069916; rs2069924; rs2069931; rs1799808; rs2069920; and rs6714364, the composition comprising two or more oligonucleotides or peptide nucleic acids each of about 10 to about 400 nucleotides and each capable of hybridizing to a sequence contained in an isolated sample from a human subject, wherein the isolated sample contains one or more of group consisting of the human subject's protein C gene sequence, a sequence complementary thereto, and a corresponding RNA sequence.

32. The composition of claim 31, wherein the two or more polymorphisms are improved response polymorphisms selected from one or more of the following: rs2069915GG; rs2069915AA; rs2069910CC; rs2069910TT; rs2069916CC; rs2069916TT; rs2069924CC; rs2069924TT; rs2069931 CC; rs2069931TT; rs1799808CC; rs1799808TT; rs2069920CC; rs2069920TT; rs6714364AA; and rs6714364TT.

33. Two or more oligonucleotides or peptide nucleic acids selected from the group consisting of:
(a) an oligonucleotide or peptide nucleic acid that hybridizes under high stringency conditions to a nucleic acid molecule comprising SEQ ID NO:1 having a G at position 301 but not to a nucleic acid molecule comprising SEQ ID NO:1 having a A at position 301;
(b) an oligonucleotide or peptide nucleic acid that hybridizes under high stringency conditions to a nucleic acid molecule comprising SEQ ID NO:1 having an A at position 301 but not to a nucleic acid molecule comprising SEQ ID NO:1 having a G at position 301;
(c) an oligonucleotide or peptide nucleic acid that hybridizes under high stringency conditions to a nucleic acid molecule comprising SEQ ID NO:2 having a C at position 301 but not to a nucleic acid molecule comprising SEQ ID NO:2 having a T at position 301;
(d) an oligonucleotide or peptide nucleic acid that hybridizes under high stringency conditions to a nucleic acid molecule comprising SEQ ID NO:2 having a T at position 301 but not to a nucleic acid molecule comprising SEQ ID NO:2 having a C at position 301;
(e) an oligonucleotide or peptide nucleic acid that hybridizes under high stringency conditions to a nucleic acid molecule comprising SEQ ID NO:3 having a C at position 301 but not to a nucleic acid molecule comprising SEQ ID NO:3 having a T at position 301;
(f) an oligonucleotide or peptide nucleic acid that hybridizes under high stringency conditions to a nucleic acid molecule comprising SEQ ID NO:3 having a T at position 301 but not to a nucleic acid molecule comprising SEQ ID NO:3 having a C at position 301;
(g) an oligonucleotide or peptide nucleic acid that hybridizes under high stringency conditions to a nucleic acid molecule comprising SEQ ID NO:4 having a C at position 301 but not to a nucleic acid molecule comprising SEQ ID NO:4 having a Tat position 301;
(h) an oligonucleotide or peptide nucleic acid that hybridizes under high stringency conditions to a nucleic acid molecule comprising SEQ ID NO:4 having a T at position 301 but not to a nucleic acid molecule comprising SEQ ID NO:4 having a C at position 301;
(i) an oligonucleotide or peptide nucleic acid that hybridizes under high stringency conditions to a nucleic acid molecule comprising SEQ ID NO:5 having a C at position 301 but not to a nucleic acid molecule comprising SEQ ID NO:5 having a T at position 301;
(j) an oligonucleotide or peptide nucleic acid that hybridizes under high stringency conditions to a nucleic acid molecule comprising SEQ ID NO:5 having a T at position 301 but not to a nucleic acid molecule comprising SEQ ID NO:5 having a C at position 301;
(k) an oligonucleotide or peptide nucleic acid that hybridizes under high stringency conditions to a nucleic acid molecule comprising SEQ ID NO:6 having a C at position 433 but not to a nucleic acid molecule comprising SEQ ID NO:6 having a T at position 433;
(l) an oligonucleotide or peptide nucleic acid that hybridizes under high stringency conditions to a nucleic acid molecule comprising SEQ ID NO:6 having a T at position 433 but not to a nucleic acid molecule comprising SEQ ID NO:6 having a C at position 433;
(m) an oligonucleotide or peptide nucleic acid that hybridizes under high stringency conditions to a nucleic acid molecule comprising SEQ ID NO:7 having a C at position 201 but not to a nucleic acid molecule comprising SEQ ID NO:7 having a T at position 201;
(n) an oligonucleotide or peptide nucleic acid that hybridizes under high stringency conditions to a nucleic acid molecule comprising SEQ ID NO:7 having a T at position 201 but not to a nucleic acid molecule comprising SEQ ID NO:7 having a C at position 201;
(o) an oligonucleotide or peptide nucleic acid that hybridizes under high stringency conditions to a nucleic acid molecule comprising SEQ ID NO:8 having a T at position 326 but not to a nucleic acid molecule comprising SEQ ID NO:8 having an A at position 326; and
(p) an oligonucleotide or peptide nucleic acid that hybridizes under high stringency conditions to a nucleic acid molecule comprising SEQ ID NO:8 having an A at position 326 but not to a nucleic acid molecule comprising SEQ ID NO:8 having a T at position 326.

34. An array of oligonucleotides or peptide nucleic acids attached to a solid support, the array comprising two or more of the oligonucleotides or peptide nucleic acids set out in claim 33.

35. The array of claim 34, wherein the oligonucleotides or peptide nucleic acids are attached to the solid support through a linker molecule.

36. A composition comprising an addressable collection of two or more oligonucleotides or peptide nucleic acids, the two or more oligonucleotides or peptide nucleic acids selected from the oligonucleotides or peptide nucleic acids set out in any one of claims 31-33.

37. A composition comprising an addressable collection of two or more oligonucleotides or peptide nucleic acids, the two or more oligonucleotides or peptide nucleic acids consisting essentially of two or more nucleic acid molecules set out in SEQ ID NOS:1-8 or complements, fragments, variants, or analogs thereof.

38. A composition comprising an addressable collection of two or more oligonucleotides or peptide nucleic acids, the two or more oligonucleotides or peptide nucleic acids consisting essentially of two or more nucleic acid molecules set out in TABLES 1C and 1D or complements, fragments, variants, or analogs thereof.

39. The oligonucleotides or peptide nucleic acids of any one of claims 31-33, further comprising one or more of the following: a detectable label; a quencher; a mobility modifier; and a contiguous non-target sequence situated 5′ or 3′ to the target sequence or 5′ and 3′ to the target sequence.

40. A computer readable medium comprising a plurality of digitally encoded genotype correlations selected from the protein C gene SNP correlations in TABLE 1E, wherein each correlation of the plurality has a value representing an indication of responsiveness to treatment with activated protein C.


SEQUENCE LISTING (8 sequences)

SEQ ID NO: 1 - DNA, 601 bp, Homo sapiens
  Variation (301)..(301): r is a or g
ggctgttggtttcatttgtgcctttatagagctgtttatctgcttgggacctgcacctccaccctttcccaaggtgccctcagctcaggcataccctcctctaggatgccttttcccccatcccttcttgctcacacccccaacttgatctctccctcctaactgtgccctgcacccaagacagacacttcacagagcccaggagacacctggggacccttcctgggtgataggtctgtctatcctccaggtgtccctgcccaaggggagaagcatggggaatacttggttgggggaggaraggaagactggggggatgtgtcaagatggggctgcacgtggtgtactggcagaagagtgagaggatttaacttggcagcctttacagcagcagccagggcttgagtacttatctctgggccagggactgtattggatgttttacatgacggtctcatccccatgtttttggatgagtaaattgaaccttagaaaggtaaagacactggctcaaggtcacacagagatcggggtggggttcacagggaggcctgtccatctcagagcaaggcttcgtcctccaactgccatctgcttcctg

SEQ ID NO: 2 - DNA, 601 bp, Homo sapiens
  variation (301)..(301): y is c or t
ccatctctctgagccctgggtgaggtgaggggcagatgggaatggcaggaatcaactgacaagtcccaggtaggccagctgccagagtgccacacaggggctgccagggcaggcatgcgtgatggcagggagccccgcgatgacctcctaaagctccctcctccacacggggatggtcacagagtcccctgggccttccctctccacccactcactccctcaactgtgaagaccccaggcccaggctaccgtccacactatccagcacagcctcccctactcaaatgcacactggcctcayggctgccctgccccaacccctttcctggtctccacagccaacgggaggaggccatgattcttggggaggtccgcaggacacatgggcccctaaagccacaccaggctgttggtttcatttgtgcctttatagagctgtttatctgcttgggacctgcacctccaccctttcccaaggtgccctcagctcaggcataccctcctctaggatgccttttcccccatcccttcttgctcacacccccaacttgatctctccctcctaactgtgccctgcacccaagacagacacttcacagag

SEQ ID NO: 3 - DNA, 601 bp, Homo sapiens
  Variation (301)..(301): y is c or t
atctgcttgggacctgcacctccaccctttcccaaggtgccctcagctcaggcataccctcctctaggatgccttttcccccatcccttcttgctcacacccccaacttgatctctccctcctaactgtgccctgcacccaagacagacacttcacagagcccaggagacacctggggacccttcctgggtgataggtctgtctatcctccaggtgtccctgcccaaggggagaagcatggggaatacttggttgggggaggagaggaagactggggggatgtgtcaagatggggctgcaygtggtgtactggcagaagagtgagaggatttaacttggcagcctttacagcagcagccagggcttgagtacttatctctgggccagggactgtattggatgttttacatgacggtctcatccccatgtttttggatgagtaaattgaaccttagaaaggtaaagacactggctcaaggtcacacagagatcggggtggggttcacagggaggcctgtccatctcagagcaaggcttcgtcctccaactgccatctgcttcctggggaggaaaagagcagaggacccctgcgccaagccat

SEQ ID NO: 4 - DNA, 601 bp, Homo sapiens
  Variation (301)..(301): y is c or t
aactctcaacatgccctcccacctgcactgccttcctggaagccccacagcctcctatggttccgtggtccagtccttcagcttctgggcgcccccatcacgggctgagatttttgctttccagtctgccaagtcagttactgtgtccatccatctgctgtcagcttctggaattgttgctgttgtgccctttccattcttttgttatgatgcagctcccctgctgacgacgtcccattgctcttttaagtctagatatctggactgggcattcaaggcccattttgagcagagtcgggcygacctttcagccctcagttctccatggagtatgcgctctcttcttggcagggaggcctcacaaacatgccatgcctattgtaggagctctccaagaatgctcacctccttctccctgtaattcctttcctctgtgaggagctcagcagcatcccattatgagaccttactaatcccagggatcacccccaacagccctggggtacaatgagcttttaagaagtttaaccacctatgtaaggagacacaggcagtgggcgatgctgcctggcctgactcttgccattgggtggtactgttt

SEQ ID NO: 5 - DNA, 601 bp, Homo sapiens
  Variation (301)..(301): y is c or t
cgatctatggcaatttctggagggggggtctggctcaactctttatgccaaaaagaaggcaaagcatattgagaaaggccaaattcacatttcctacagcataatctatggccagtggccccccgtggggcttggcttagaattcccaggtgctcttcccagggaaccatcagtctggactgagaggaccttctctctcaggtgggacccggccctgtcctccctggcagtgccgtgttctgggggtcctcctctctgggtctcactgcccctggggtctctccagctacctttgctccaygttcctttgtggctctggtctgtgtctggggtttccaggggtctcgggcttccctgctgcccattccttctctggtctcacggctccgtgactcctgaaaaccaaccagcatcctacctctttgggattgacacctgttggccactccttctggcaggaaaagtcaccgttgatagggttccacggcatagacaggtggctccgcgccagtgcctgggacgtgtgggtgcacagtctccgggtgaaccttcttcaggccctctgcccaggcctgcaggggcacagcagtgggtgggcctc

SEQ ID NO: 6 - DNA, 633 bp, Homo sapiens
  Variation (433)..(433): y is c or t
aaaaatagaggttagagggatgctatgtgccattgtgtgtgtgtgttgggggtggggattgggggtgatttgtgagcaattggaggtgagggtggagcccagtgcccagcacctatgcactggggacccaaaaaggagcatcttctcatgattttatgtatcagaaattgggatggcatgtcattgggacagcgtcttttttcttgtatggtggcacataaatacatgtgtcttataattaatggtattttagatttgacgaaatatggaatattacctgttgtgctgatcttgggcaaactataatatctctgggcaaaaatgtccccatctgaaaaacagggacaacgttcctccctcagccagccactatggggctaaaatgagaccacatctgtcaagggttttgccctcacctccctccctgctggayggcatccttggtgggcagaggtgggcttcgggcagaacaagccgtgctgagctaggaccaggagtgctagtgccactgtttgtctatggagagggaggcctcagtgctgagggccaagcaaatatttgtggttatggattaactcgaactccaggctgtcatggcggcaggacggcgaacttgcagtatctccacgaccc

SEQ ID NO: 7 - DNA, 401 bp, Homo sapiens
  Variation (201)..(201): y is c or t
tcacatcaaagggagaaaatctgattgttcagggggtcggaagacagggtctgtgtcctatttgtctaagggtcagagtcctttggagcccccagagtcctgtggacgtggccctaggtagtagggtgagcttggtaacggggctggcttcctgagacaaggctcagacccgctctgtccctggggatcgcttcagccacyaggacctgaaaattgtgcacgcctgggcccccttccaaggcatccagggatgctttccagtggaggctttcagggcaggagaccctctggcctgcaccctctcttgccctcagcctccacctccttgactggacccccatctggacctccatccccaccacctctttccccagtggcctccctggcagacaccacagtga

SEQ ID NO: 8 - DNA, 526 bp, Homo sapiens
  Variation (326)..(326): w is a or t
cttctgcaggcagggagaggggagtcaagtcagtgaggagggctttcgcagtttctcttacaaactctcaacatgccctcccacctgcactgccttcctggaagccccacagcctcctatggttccgtggtccagtccttcagcttctgggcgcccccatcacgggctgagatttttgctttccagtctgccaagtcagttactgtgtccatccatctgctgtcagcttctggaattgttgctgttgtgccctttccattcttttgttatgatgcagctcccctgctgacgacgtcccattgctcttttaagtctagatatctggwctgggcattcaaggcccattttgagcagagtcgggccgacctttcagccctcagttctccatggagtatgcgctctcttcttggcagggaggcctcacaaacatgccatgcctattgtaggagctctccaagaatgctcacctccttctccctgtaattcctttcctctgtgaggagctcagcagcatcccattatgaga